(19) United States
(12) Patent Application Publication
(10) Pub. No.: US 2014/0051665 A1
(43) Pub. Date: Feb. 20, 2014

(54) PROSTATE CANCER THERAPY WITH HSP90 INHIBITORY COMPOUNDS

(71) Applicants: David Proia, Newton, MA (US); Suqin He, West Roxbury, MA (US)

(72) Inventors: David Proia, Newton, MA (US); Suqin He, West Roxbury, MA (US)

(73) Assignee: SYNTA PHARMACEUTICALS CORP., Lexington, MA (US)

(21) Appl. No.: 14/001,465

(22) PCT Filed: Feb. 23, 2012
(86) PCT No.: PCT/US12/26268

§ 371 Date: Oct. 31, 2013

Related U.S. Application Data
(60) Provisional application No. 61/446,099, filed on Feb. 24, 2011.
(60) Provisional application No. 61/491,531, filed on May 31, 2011.

(51) Int. Cl. A61K 31/4196; C07F 9/6518; C07D 403/10; A61K 31/675; A61K 31/337

(52) U.S. Cl. CPC A61K 31/4196; A61K 31/675; A61K 31/337; C07D 403/10; C07F 9/65185
USPC 514/80; 514/384

(57) ABSTRACT

Method for treating a subject with prostate cancer, comprising administering to the an effective amount of a compound represented by the following structural formula: a tautomer, or a pharmaceutically acceptable salt thereof. The variables depicted in the structural formula are defined herein.

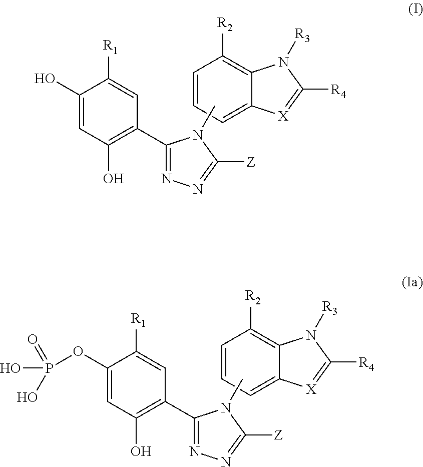

### CROSS-REFERENCE TO RELATED PATENT APPLICATIONS

This application claims the benefit of priority to U.S. Provisional Patent Application Nos. 61/446,099, filed on Feb. 24, 2011, and. 61/491,531, filed on May 31, 2011. The contents of each of these applications are incorporated herein by reference in their entireties.

### BACKGROUND OF THE INVENTION

Although tremendous advances have been made in elucidating the genomic abnormalities that cause malignant cancer cells, currently available chemotherapy remains unsatisfactory, and the prognosis for the majority of patients diagnosed with cancer remains dismal. Most chemotherapeutic agents act on a specific molecular target thought to be involved in the development of the malignant phenotype. However, a complex network of signaling pathways regulate cell proliferation and the majority of malignant cancers are facilitated by multiple genetic abnormalities in these pathways. Therefore, it is less likely that a therapeutic agent that acts on one molecular target will be fully effective in curing a patient who has cancer.

Heat shock proteins (HSPs) are a class of chaperone proteins that are up-regulated in response to elevated temperature and other environmental stresses, such as ultraviolet light, nutrient deprivation and oxygen deprivation. HSPs act as chaperones to other cellular proteins (called client proteins), facilitate their proper folding and repair and aid in the refolding of misfolded client proteins. There are several known families of HSPs, each having its own set of client proteins. The Hsp90 family is one of the most abundant HSP families accounting for about 1-2% of proteins in a cell that is not under stress and increasing to about 4-6% in a cell under stress. Inhibition of Hsp90 results in the degradation of its client proteins via the ubiquitin proteasome pathway. Unlike other chaperone proteins, the client proteins of Hsp90 are mostly protein kinases or transcription factors involved in signal transduction, and a number of its client proteins have been shown to be involved in the progression of cancer.

### SUMMARY OF THE INVENTION

It is now found that certain triazolone Hsp90 inhibitors are particularly effective in treating patients with prostate cancer, such as metastatic prostate cancer, metastatic hormone-resistant prostate cancer or metastatic castration-resistant prostate cancer, or prostate cancer wherein it was previously treated with docetaxel-based chemotherapy.

The present method utilizes Hsp90 inhibitory compounds of formula (I) or (Ia) or compounds in Tables 1 or 2, or pharmaceutically acceptable salts or tautomers thereof, for the treatment of prostate cancer.

In one embodiment, the method includes the step of administering to a subject with prostate cancer an amount of from about 2 mg/m² to about 500 mg/m² of the triazolone compound of formula (I) or (Ia) or a compound in Tables 1 or 2. In one embodiment, the compound of formula (I) or (Ia) or in Tables 1 or 2 may be administered weekly. In one embodiment, the compound of formula (I) or (Ia) or in Tables 1 or 2 may be administered twice weekly. In one embodiment, the compound may be administered for about 3 weeks. In another embodiment, the twice weekly administration for 3 weeks may be repeated after about 7 days dose-free. In one embodiment, the twice weekly administration after 7 days dose free may be repeated two or more times. In any one of these embodiments, the triazolone compound may be 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof. In any one of these embodiments, the triazolone compound may be 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof.

It is also found that certain triazolone Hsp90 inhibitors and taxane combinations are surprisingly effective at treating subjects with prostate cancer. The particular combination therapies disclosed herein demonstrate surprising biological activity by showing significant anticancer effects with minimal side effects. In one embodiment, the combination includes the triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, and a taxane. In one embodiment, the combination includes the triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, and docetaxel. In one embodiment, the combination includes the triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, and a taxane. In one embodiment, the combination includes the triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, and docetaxel.

In another embodiment, the combination method of treating a subject with prostate cancer includes the step of administering to the subject an effective amount of an Hsp90 inhibitor described herein and a taxane. In one embodiment, the administration of the Hsp90 inhibitor and the taxane are done concurrently. In another embodiment, the administration of the Hsp90 inhibitor and the taxane are done sequentially. In any one of these embodiments, the taxane may be docetaxel, paclitaxel or Abraxane®. In any one of these embodiments, the Hsp90 inhibitor is a compound represented by formula (I) or (Ia) or a compound in Table 1 or Table 2. In any one of these embodiments, the Hsp90 inhibitor may be the triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof. In any one of these embodiments, the Hsp90 inhibitor may be the triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof.

In one embodiment, the method includes the use of an Hsp90 inhibitor described herein for the manufacture of a medicament for treating prostate cancer in combination with a taxane.

In certain embodiments, the methods described herein provide compositions of Hsp90 inhibitory compounds described herein with a taxane for the treatment of prostate cancer in a subject in need thereof.

In another embodiment, the method also includes monitoring the treatment response of a subject with prostate cancer being treated with a compound of formula (I) or (Ia) or a compound in Tables 1 or 2, comprising (a) determining the level of maspin in a biological sample derived from the subject before the treatment of said compound; (b) determining the level of maspin in a biological sample derived from the subject at a time point during or after administration of the compound; and (c) comparing the level of maspin in the biological sample derived from the subject during or after treatment with that before the treatment, wherein an increase of the maspin level in the biological sample is indicative of a positive response to the treatment of the triazolone compound. In one embodiment, the subject may be treated with the triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof. In one embodiment, the subject may be treated with the triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof.

### BRIEF DESCRIPTION OF THE FIGURES

FIG. 1 shows the cell cycle analysis for PC3 and DU145 after treatment with ganetespib (Compound 1) or 17-AAG.

FIG. 2 shows ganetespib or 17-AAG induced apoptosis at different concentrations in PCa cells.

FIG. 3 shows prostate cancer cells were treated with ganetespib or 17-AAG for 24 hr and analyzed by microscopy (left panel), annexin VI staining by FACS, or Western blot for expression of total/phosphor MCL-1, an antiapoptotic Bcl-2 family protein.

FIG. 4 shows that treatment of ganetespib or 17-AAG for 24 hr disrupted multiple oncogenic Hsp90 client proteins of LNCaP cells which were AR-dependent prostate cancer cells.

FIG. 5 shows that treatment of ganetespib or 17-AAG for 24 hr disrupted multiple oncogenic Hsp90 client proteins of 22Rv1 cells which were AR-dependent prostate cancer cells.

FIG. 6 shows that treatment of ganetespib or 17-AAG for 24 hr disrupted multiple oncogenic Hsp90 client proteins of DU145 cells which was AR-independent prostate cancer cells.

FIG. 7 showed the kinetic response of Hsp90 client proteins after treatment with ganetespib or 17-AAG for indicated amount of drug and time in prostate cancer cells of LNCaP.

FIG. 8 shows the kinetic response of Hsp90 client proteins after treatment with ganetespib or 17-AAG for indicated amount of drug and time in prostate cancer cells of DU145.

FIG. 9 shows the kinetic response of Hsp90 client proteins after treatment with ganetespib or 17-AAG for indicated amount of drug and time in prostate cancer cells of PC3.

FIG. 10 shows that ganetespib destabilized the master cell cycle regulator CDK1 and the DNA damage checkpoint CHK1.

FIG. 11 shows that inhibition of CHK signaling by AZD-7762 was in synergy with ganetespib in killing PC3 cells.

FIG. 12 shows PC3 xenografts were implanted in nude mice, followed by treatment with ganetespib once a week at 150 mg/kg or twice a week at 50 mg/kg for 4 weeks. Ganetespib displayed potent single agent activity versus vehicle, with % T/C values of 17, 3 respectively.

FIG. 13 shows that inhibition of Hsp90 by ganetespib disrupts intrinsic and cytokine mediated activation of the JAK/STAT pathway as well as mitogenic signaling in DU-145 prostate cancer cells. DU-145 prostate cancer cells were treated with ganetespib as shown, in the absence or presence of IL-6, and analyzed by Western blot.

FIG. 14 shows that ganetespib displays potent single agent activity.

FIG. 15 shows DU-145 cells treated with ganetespib, docetaxel or the combination of the two for 72 hr, and cell viability determination by alamarBlue, indicating significant benefits of the combination treatment.

FIG. 16 shows LNCaP cells treated with ganetespib, docetaxel or the combination of the two for one hour on day one and day two. Twenty-four hours later, viability was assessed by microscopy. Short exposure to both drugs displays considerably greater cell death than either agent alone.

FIG. 17 is a normalized isobologram for the concurrent treatment of docetaxel with Ganetespib in DU-145 cells, indicating synergistic effect of the combination treatment.

FIG. 18 illustrates LNCaP cells cultured in charcoal-stripped medium for 24 h and then treated with 250 nM ganetespib, 1 μM geldanamycin (GA), or vehicle for 24 h in the absence or presence of 10 nM androgen (R1881).

FIG. 19 shows 22Rv1 cells treated with ganetespib at 0, 10 or 25 nM for 24 h. Cell lysates were immunoblotted using antibodies against AR, phosphorylated and total AKT, PARP and GAPDH. Expression of the full length AR and truncated V7 receptor isoform are indicated by arrowheads.

FIG. 20 shows HeLa cells transiently transfected with 3 ng of pCR3.1-AR or 0.5 ng of pCR3.1-ARV7 plasmid to induce expression of the full length and V7 truncated AR proteins, respectively. Twenty four hours following infection, cells were treated with 10 nM R1881, 1 mM GA, or 250 nM ganetespib as indicated. Cell lysates were resolved by SDS-PAGE and immunoblotted with an anti-AR antibody. Total protein levels were determined using an anti-actin antibody.

### DETAILED DESCRIPTION OF THE INVENTION

Unless otherwise specified, the below terms used herein are defined as follows:

As used herein, the term “alkyl” means a saturated, straight chain or branched, non-cyclic hydrocarbon having from 1 to 10 carbon atoms. Representative straight chain alkyls include methyl, ethyl, n-propyl, n-butyl, n-pentyl, n-hexyl, n-heptyl, n-octyl, n-nonyl and n-decyl; while representative branched alkyls include isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimtheylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl, and the like. The term “(C₁-C₆)alkyl” means a saturated, straight chain or branched, non-cyclic hydrocarbon having from 1 to 6 carbon atoms. Alkyl groups included in compounds described herein may be optionally substituted with one or more substituents.

As used herein, the term “alkenyl” means a straight chain or branched, non-cyclic hydrocarbon having from 2 to 10 carbon atoms and having at least one carbon-carbon double bond. Representative straight chain and branched (C₂-C₁₀)alkenyls include vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl, 3-decenyl, and the like. Alkenyl groups included in compounds described herein may be optionally substituted with one or more substituents.

As used herein, the term “alkynyl” means a straight chain or branched, non-cyclic hydrocarbon having from 2 to 10 carbon atoms and having at least one carbon-carbon triple bond. Representative straight chain and branched alkynyls include acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl, 9-decynyl, and the like. Alkynyl groups included in compounds described herein may be optionally substituted with one or more substituents.

As used herein, the term “cycloalkyl” means a saturated, mono- or polycyclic, non-aromatic hydrocarbon having from 3 to 20 carbon atoms. Representative cycloalkyls include cyclopropyl, 1-methylcyclopropyl, cyclobutyl, cyclopentyl, cyclohexyl, cycloheptyl, cyclooctyl, cyclononyl, cyclodecyl, octahydropentalenyl, and the like. Cycloalkyl groups included in compounds described herein may be optionally substituted with one or more substituents.

As used herein, the term “cycloalkenyl” means a mono- or polycyclic, non-aromatic hydrocarbon having at least one carbon-carbon double bond in the cyclic system and having from 3 to 20 carbon atoms. Representative cycloalkenyls include cyclopentenyl, cyclopentadienyl, cyclohexenyl, cyclohexadienyl, cycloheptenyl, cycloheptadienyl, cycloheptatrienyl, cyclooctenyl, cyclooctadienyl, cyclooctatrienyl, cyclooctatetraenyl, cyclononenyl, cyclononadienyl, cyclodecenyl, cyclodecadienyl, 1,2,3,4,5,8-hexahydronaphthalenyl, and the like. Cycloalkenyl groups included in compounds described herein may be optionally substituted with one or more substituents.

As used herein, the term “alkylene” refers to an alkyl group that has two points of attachment. The term “(C₁-C₆)alkylene” refers to an alkylene group that has from one to six carbon atoms. Straight chain (C₁-C₆)alkylene groups are preferred. Non-limiting examples of alkylene groups include methylene (—CH₂—), ethylene (—CH₂CH₂—), n-propylene (—CH₂CH₂CH₂—), isopropylene (—CH₂CH(CH₃)—), and the like. Alkylene groups included in compounds described herein may be optionally substituted with one or more substituents.

As used herein, the term “lower” refers to a group having up to four atoms. For example, a “lower alkyl” refers to an alkyl radical having from 1 to 4 carbon atoms, “lower alkoxy” refers to “—O—(C₁-C₄)alkyl and a “lower alkenyl” or “lower alkynyl” refers to an alkenyl or alkynyl radical having from 2 to 4 carbon atoms.

As used herein, the term “haloalkyl” means an alkyl group, in which one or more, including all, the hydrogen radicals are replaced by a halo group(s), wherein each halo group is independently selected from —F, —Cl, —Br, and —I. For example, the term “halomethyl” means a methyl in which one to three hydrogen radical(s) have been replaced by a halo group. Representative halo alkyl groups include trifluoromethyl, bromomethyl, 1,2-dichloroethyl, 4-iodobutyl, 2-fluoropentyl, and the like.

As used herein, an “alkoxy” is an alkyl group which is attached to another moiety via an oxygen linker. Alkoxy groups included in compounds described herein may be optionally substituted with one or more substituents.

As used herein, a “haloalkoxy” is a haloalkyl group which is attached to another moiety via an oxygen linker.

As used herein, the term an “aromatic ring” or “aryl” means a mono- or polycyclic hydrocarbon, containing from 6 to 15 carbon atoms, in which at least one ring is aromatic. Examples of suitable aryl groups include phenyl, tolyl, anthracenyl, fluorenyl, indenyl, azulenyl, and naphthyl, as well as benzo-fused carbocyclic moieties such as 5,6,7,8-tetrahydronaphthyl. Aryl groups included in compounds described herein may be optionally substituted with one or more substituents. In one embodiment, the aryl group is a monocyclic ring, wherein the ring comprises 6 carbon atoms, referred to herein as “(C₆)aryl.”

As used herein, the term “aralkyl” means an aryl group that is attached to another group by a (C₁-C₆)alkylene group. Representative aralkyl groups include benzyl, 2-phenyl-ethyl, naphth-3-yl-methyl and the like. Aralkyl groups included in compounds described herein may be optionally substituted with one or more substituents.

As used herein, the term “heterocyclyl” means a monocyclic or a polycyclic, saturated or unsaturated, non-aromatic ring or ring system which typically contains 5- to 20-members and at least one heteroatom. A heterocyclic ring system can contain saturated ring(s) or unsaturated non-aromatic ring(s), or a mixture thereof. A 3- to 10-membered heterocycle can contain up to 5 heteroatoms, and a 7- to 20-membered heterocycle can contain up to 7 heteroatoms. Typically, a heterocycle has at least one carbon atom ring member. Each heteroatom is independently selected from nitrogen, which can be oxidized (e.g., N(O)) or quaternized, oxygen and sulfur, including sulfoxide and sulfone. The heterocycle may be attached via any heteroatom or carbon atom. Representative heterocycles include morpholinyl, thiomorpholinyl, pyrrolidinonyl, pyrrolidinyl, piperidinyl, piperazinyl, hydantoinyl, valerolactamyl, oxiranyl, oxetanyl, tetrahydrofuranyl, tetrahydropyranyl, tetrahydropyrindinyl, tetrahydropyrimidinyl, tetrahydrothiophenyl, tetrahydrothiopyranyl, and the like. A heteroatom may be substituted with a protecting group known to those of ordinary skill in the art, for example, a nitrogen atom may be substituted with a tert-butoxycarbonyl group. Furthermore, the heterocyclyl included in compounds described herein may be optionally substituted with one or more substituents. Only stable isomers of such substituted heterocyclic groups are contemplated in this definition.

As used herein, the term “heteroaromatic”, “heteroaryl”, or like terms, means a monocyclic or a polycyclic, unsaturated radical containing at least one heteroatom, in which at least one ring is aromatic. Polycyclic heteroaryl rings must contain at least one heteroatom, but not all rings of a polycyclic heteroaryl moiety must contain heteroatoms. Each heteroatom is independently selected from nitrogen, which can be oxidized (e.g., N(O)) or quaternized, oxygen and sulfur, including sulfoxide and sulfone. Representative heteroaryl groups include pyridyl, 1-oxo-pyridyl, furanyl, benzo[1,3]dioxolyl, benzo[1,4]dioxinyl, thienyl, pyrrolyl, oxazolyl, imidazolyl, thiazolyl, a isoxazolyl, quinolinyl, pyrazolyl, isothiazolyl, pyridazinyl, pyrimidinyl, pyrazinyl, a triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, indolyl, tetrahydroindolyl, azaindolyl, imidazopyridyl, quinazolinyl, purinyl, pyrrolo[2,3]pyrimidinyl, pyrazolo[3,4]pyrimidinyl, imidazo[1,2-a]pyridyl, and benzothienyl. In one embodiment, the heteroaromatic ring is selected from 5-8 membered monocyclic heteroaryl rings. The point of attachment of a heteroaromatic or heteroaryl ring may be at either a carbon atom or a heteroatom. Heteroaryl groups included in compounds described herein may be optionally substituted with one or more substituents. As used herein, the term “(C₅)heteroaryl” means an heteroaromatic ring of 5 members, wherein at least one carbon atom of the ring is replaced with a heteroatom, such as, for example, oxygen, sulfur or nitrogen. Representative (C₅)heteroaryls include furanyl, thienyl, pyrrolyl, oxazolyl, imidazolyl, thiazolyl, isoxazolyl, pyrazolyl, isothiazolyl, pyrazinyl, triazolyl, thiadiazolyl, and the like. As used herein, the term “(C₆)heteroaryl” means an aromatic heterocyclic ring of 6 members, wherein at least one carbon atom of the ring is replaced with a heteroatom such as, for example, oxygen, nitrogen or sulfur. Representative (C₆)heteroaryls include pyridyl, pyridazinyl, pyrazinyl, triazinyl, tetrazinyl, and the like.

As used herein, the term “heteroaralkyl” means a heteroaryl group that is attached to another group by a (C₁-C₆)alkylene. Representative heteroaralkyls include 2-(pyridin-4-yl)-propyl, 2-(thien-3-yl)-ethyl, imidazol-4-yl-methyl, and the like. Heteroaralkyl groups included in compounds described herein may be optionally substituted with one or more substituents.

As used herein, the term “halogen” or “halo” means —F, —Cl, —Br or —I.

As used herein the term “heteroalkyl” means a straight or branched alkyl group wherein one or more of the internal carbon atoms in the chain is replaced by a heteroatom. For example, a heteroalkyl is represented by the formula —[CH₂]ₓ—Z—[CH₂]_{y}[CH₃], wherein x is a positive integer and y is zero or a positive integer, Z is O, NR, S, S(O), or S(O)₂, and wherein replacement of the carbon atom does not result in a unstable compound. Heteroalkyl groups included in compounds described herein may be optionally substituted with one or more substituents.

Suitable substituents for an alkyl, alkylene, alkenyl, alkynyl, cycloalkyl, cycloalkenyl, heterocyclyl, aryl, aralkyl, heteroaryl, and heteroaralkyl groups include are those substituents which form a stable compound described herein without significantly adversely affecting the reactivity or biological activity of the compound described herein. Examples of substituents for an alkyl, alkylene, alkenyl, alkynyl, cycloalkyl, cycloalkenyl, heterocyclyl, aryl, aralkyl, heteroaryl, and heteroaralkyl include an alkyl, alkenyl, alkynyl, cycloalkyl, cycloalkenyl, heterocyclyl, aryl, heteroaryl, aralkyl, heteraralkyl, heteroalkyl, alkoxy, (each of which can be optionally and independently substituted), —C(O)NR²⁸R²⁹, —C(S)NR²⁸R²⁹, —C(NR⁴²)NR²⁸R²⁹, —NR⁴³C(O)R⁴¹, —NR⁴³C(S)R⁴¹, —NR⁴³C(NR⁴²)R⁴¹, halo, —OR⁴³, cyano, nitro, —C(O)R⁴³, —C(S)R⁴³, —C(NR⁴²)R⁴³, —NR²⁸R²⁹, —C(O)OR⁴³, —C(S)OR⁴³, —C(NR⁴²)OR⁴³, —OC(O)R⁴³, —OC(S)R⁴³, —OC(NR⁴²)R⁴³, —NR⁴⁰C(O)NR²⁸R²⁹, —NR⁴³C(S)NR²⁸R²⁹, —NR⁴³C(NR⁴²)NR²⁸R²⁹, —OC(O)NR²⁸R²⁹, —OC(S)NR²⁸R²⁹, —OC(NR⁴²)NR²⁸R²⁹, —NR⁴³C(O)OR⁴¹, —NR⁴³C(S)OR⁴¹, —NR⁴³C(NR⁴²)OR⁴¹, —S(O)ₖR⁴³, —OS(O)ₖR⁴³, —NR⁴³S(O)ₖR⁴³, —S(O)ₖNR²⁸R²⁹, —OS(O)ₖNR²⁸R²⁹, —NR⁴³S(O)ₖNR²⁸R²⁹, guanidino, —C(O)SR⁴¹, —C(S)SR⁴¹, —C(NR⁴²)SR⁴¹, —OC(O)OR⁴¹, —OC(S)OR⁴¹, —OC(NR⁴²)OR⁴¹, —SC(O)R⁴³, —SC(O)OR⁴¹, —SC(NR⁴²)OR⁴¹, —SC(S)R⁴³, SC(S)OR⁴¹, —SC(O)NR²⁸R²⁹, —SC(NR⁴²)NR²⁸R²⁹, —SC(S)NR²⁸R²⁹, —SC(NR⁴²)R⁴³, —OS(O)ₖOR⁴¹, —S(O)ₖOR⁴¹, —NR⁴⁰OS(O)ₖOR⁴¹, —SS(O)ₖR⁴³, —SS(O)ₖOR⁴¹, —SS(O)ₖNR²⁸R²⁹, —OP(O)(OR⁴¹)₂, or —SP(O)(OR⁴¹)₂. In addition, any saturated portion of an alkyl, cycloalkyl, alkylene, heterocyclyl, alkenyl, cycloalkenyl, alkynyl, aralkyl and heteroaralkyl groups, may also be substituted with ═O, ═S, or ═N—R⁴².

Each R²⁸, R²⁹, and R⁴⁰ is independently H, alkyl, alkenyl, alkynyl, cycloalkyl, cycloalkenyl, heterocyclyl, aryl, heteroaryl, aralkyl, or heteraralkyl, wherein each alkyl, alkenyl, alkynyl, cycloalkyl, cycloalkenyl, heterocyclyl, aryl, heteroaryl, aralkyl, or heteroalkyl represented by R²⁸, R²⁹, or R⁴⁰ is optionally and independently substituted.

Each R⁴¹ and R⁴³ is independently H, alkyl, alkenyl, alkynyl, cycloalkyl, cycloalkenyl, heterocyclyl, aryl, heteroaryl, aralkyl, or heteraralkyl, wherein each alkyl, alkenyl, alkynyl, cycloalkyl, cycloalkenyl, heterocyclyl, aryl, heteroaryl, aralkyl, and heteraralkyl represented by R⁴¹ or R⁴³ is optionally and independently unsubstituted.

Each R⁴² is independently H, alkyl, alkenyl, alkynyl, cycloalkyl, cycloalkenyl, heterocyclyl, aryl, heteroaryl, aralkyl, heteraralkyl, —C(O)R⁴³, —C(O)NR²⁸R²⁹, —S(O)ₚR⁴³, or —S(O)ₚNR²⁸R²⁹, wherein each alkyl, alkenyl, alkynyl, cycloalkyl, cycloalkenyl, heterocyclyl, aryl, heteroaryl, aralkyl and heteraralkyl represented by R⁴² is optionally and independently substituted.

The variable k is 0, 1 or 2.

When a heterocyclyl, heteroaryl or heteroaralkyl group contains a nitrogen atom, it may be substituted or unsubstituted. When a nitrogen atom in the aromatic ring of a heteroaryl group has a substituent, the nitrogen may be oxidized or a quaternary nitrogen.

The compounds described herein are defined herein by their chemical structures and/or chemical names. Where a compound is referred to by both a chemical structure and a chemical name, and the chemical structure and chemical name conflict, the chemical structure is determinative of the compound's identity.

Only those choices and combinations of substituents that result in a stable structure are contemplated. Such choices and combinations will be apparent to those of ordinary skill in the art and may be determined without undue experimentation.

As used herein, the terms “subject”, “patient” and “mammal” are used interchangeably. The terms “subject” and “patient” refer to an animal (e.g., a bird such as a chicken, quail or turkey, or a mammal), preferably a mammal including a non-primate (e.g., a cow, pig, horse, sheep, rabbit, guinea pig, rat, cat, dog, and mouse) and a primate (e.g., a monkey, chimpanzee and a human), and more preferably a human. In one embodiment, the subject is a non-human animal such as a farm animal (e.g., a horse, cow, pig or sheep), or a pet (e.g., a dog, cat, guinea pig or rabbit). In a preferred embodiment, the subject is a human.

As used herein, the term “compound(s) of this invention” “triazolone compound”, or similar terms refers to a compound of any one of formulae (I) or (Ia) or a compound in Table 1 or Table 2, or a pharmaceutically acceptable salt thereof.

As used herein, the term “pharmaceutically acceptable salt” refers to a salt prepared from a compound of any one of formulae (I) or (Ia) or a compound in Table for Table 2 having an acidic functional group, such as a carboxylic acid functional group, and a pharmaceutically acceptable inorganic or organic base. Suitable bases include hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl, N-ethylamine; diethylamine; triethylamine; mono-, bis-, or tris-(2-hydroxy-lower alkyl amines), such as mono-, bis-, or tris-(2-hydroxyethyl)amine, 2-hydroxy-tert-butylamine, or tris-(hydroxymethyl)methylamine, N,N,-di-lower alkyl-N-(hydroxy lower alkyl)-amines, such as N,N-dimethyl-N-(2-hydroxyethyl)amine, or tri-(2-hydroxyethyl)amine; N-methyl-D-glucamine; and amino acids such as arginine, lysine, and the like. The term “pharmaceutically acceptable salt” also refers to a salt prepared from a compound of any one of formulae (I) or (Ia) or a compound in Table 1 or Table 2 having a basic functional group, such as an amine functional group, and a pharmaceutically acceptable inorganic or organic acid. Suitable acids include hydrogen sulfate, citric acid, acetic acid, oxalic acid, hydrochloric acid (HCl), hydrogen bromide (HBr), hydrogen iodide (HI), nitric acid, hydrogen bisulfide, phosphoric acid, isonicotinic acid, oleic acid, tannic acid, pantothenic acid, saccharic acid, lactic acid, salicylic acid, tartaric acid, bitartratic acid, ascorbic acid, succinic acid, maleic acid, besylic acid, fumaric acid, gluconic acid, glucaronic acid, formic acid, benzoic acid, glutamic acid, methanesulfonic acid, ethanesulfonic acid, benzenesulfonic acid, pamoic acid and p-toluenesulfonic acid.

A pharmaceutically acceptable carrier may contain inert ingredients which do not unduly inhibit the biological activity of the compound(s) described herein. The pharmaceutically acceptable carriers should be biocompatible, i.e., non-toxic, non-inflammatory, non-immunogenic and devoid of other undesired reactions upon the administration to a subject. Standard pharmaceutical formulation techniques can be employed, such as those described in REMINGTON, J. P., REMINGTON'S PHARMACEUTICAL SCIENCES (Mack Pub. Co., 17^{th} ed., 1985). Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate, and the like. Methods for encapsulating compositions, such as in a coating of hard gelatin or cyclodextran, are known in the art. See BAKER, ET AL., CONTROLLED RELEASE OF BIOLOGICAL ACTIVE AGENTS, (John Wiley and Sons, 1986).

As used herein, the term “effective amount” refers to an amount of a compound described herein which is sufficient to reduce or ameliorate the severity, duration, progression, or onset of a disease or disorder, delay onset of a disease or disorder, retard or halt the advancement of a disease or disorder, cause the regression of a disease or disorder, prevent or delay the recurrence, development, onset or progression of a symptom associated with a disease or disorder, or enhance or improve the therapeutic effect(s) of another therapy. The precise amount of compound administered to a subject will depend on the mode of administration, the type and severity of the disease or condition and on the characteristics of the subject, such as general health, age, sex, body weight and tolerance to drugs. For example, for a proliferative disease or disorder, determination of an effective amount will also depend on the degree, severity and type of cell proliferation. The skilled artisan will be able to determine appropriate dosages depending on these and other factors. When co-administered with other therapeutic agents, e.g., when co-administered with an anti-cancer agent, an “effective amount” of any additional therapeutic agent(s) will depend on the type of drug used. Suitable dosages are known for approved therapeutic agents and can be adjusted by the skilled artisan according to the condition of the subject, the type of condition(s) being treated and the amount of a compound described herein being used. In cases where no amount is expressly noted, an effective amount should be assumed. Non-limiting examples of an effective amount of a compound described herein are provided herein below. In a specific embodiment, a method of treating, managing, or ameliorating prostate cancer, or one or more symptoms thereof, including administering to a subject in need thereof a dose of at least 25 mg/kg, at least 50 mg/kg, at least 75 mg/kg, at least 100 mg/kg, at least 125 mg/kg, at least 150 mg/kg, or at least 200 mg/kg or more of one or more compounds described herein once every day, once every 2 days, once every 3 days, once every 4 days, once every 5 days, once every 6 days, once every 7 days, once every 8 days, once every 10 days, once every two weeks, once every three weeks, or once a month. The daily dose can be administered in a single portion. Alternatively, the daily dose can be divided into portions (typically equal portions) administered two times, three times, four times or more per day.

The dosage of a therapeutic agent other than a compound of the triazolone compound described herein, which has been or is currently being used to treat, manage, or ameliorate prostate cancer, or one or more symptoms thereof, can be used in the combination therapies of the invention. Preferably, the dosage of each individual therapeutic agent used in the combination therapy is lower than the dose of an individual therapeutic agent when given independently to treat, manage, or ameliorate a disease or disorder, or one or more symptoms thereof. The recommended dosages of therapeutic agents currently used for the treatment, management, or amelioration of prostate cancer, or one or more symptoms thereof, can obtained from any reference in the art. See, e.g., GOODMAN & GILMAN'S THE PHARMACOLOGICAL BASIS OF BASIS OF THERAPEUTICS 9^{TH} ED, (Hardman, et al., Eds., NY:Mc-Graw-Hill (1996)); PHYSICIAN'S DESK REFERENCE 57^{TH} ED. (Medical Economics Co., Inc., Montvale, N.J. (2003)).

As used herein, the terms “treat”, “treatment” and “treating” refer to the reduction or amelioration of the progression, severity and/or duration of a disease or disorder, delay of the onset of a disease or disorder, or the amelioration of one or more symptoms (preferably, one or more discernible symptoms) of a disease or disorder, resulting from the administration of one or more therapies (e.g., one or more therapeutic agents such as a compound described herein). The terms “treat”, “treatment” and “treating” also encompass the reduction of the risk of developing a disease or disorder, and the delay or inhibition of the recurrence of a disease or disorder. In specific embodiments, the terms “treat”, “treatment” and “treating” refer to the amelioration of at least one measurable physical parameter of a disease or disorder, such as growth of a tumor, not necessarily discernible by the patient. In other embodiments the terms “treat”, “treatment” and “treating” refer to the inhibition of the progression of a disease or disorder, e.g., prostate cancer, either physically by the stabilization of a discernible symptom, physiologically by the stabilization of a physical parameter, or both. In another embodiment, the terms “treat”, “treatment” and “treating” of a proliferative disease or disorder refers to the reduction or stabilization of tumor size or cancerous cell count, and/or delay of tumor formation.

As used herein, the terms “therapeutic agent” and “therapeutic agents” refer to any agent(s) that can be used in the treatment of a disease or disorder, e.g. cancer, or one or more symptoms thereof. In certain embodiments, the term “therapeutic agent” refers to a compound described herein. In certain other embodiments, the term “therapeutic agent” does not refer to a compound described herein. Preferably, a therapeutic agent is an agent that is known to be useful for, or has been or is currently being used for the treatment of a disease or disorder, e.g., cancer, or one or more symptoms thereof.

As used herein, the term “synergistic” refers to a combination of a compound described herein and another therapeutic agent, which, when taken together, is more effective than the additive effects of the individual therapies. A synergistic effect of a combination of therapies (e.g., a combination of therapeutic agents) permits the use of lower dosages of one or more of the therapeutic agent(s) and/or less frequent administration of the agent(s) to a subject with a disease or disorder, e.g., cancer. The combination therapy of triazolone compounds described herein with a taxane may permit, among other things, less frequent administration of the therapies. The ability to utilize lower dosage of one or more therapeutic agent and/or to administer the therapeutic agent less frequently may reduce the toxicity associated with the administration of the agent to a subject without reducing the efficacy of the therapy in the treatment of a disease or disorder. In addition, a synergistic effect can result in improved efficacy of agents in the prevention, management or treatment of a disease or disorder, e.g. cancer. Finally, a synergistic effect of a combination of therapies may avoid or reduce adverse or unwanted side effects associated with the use of either therapeutic agent alone.

As used herein, the term “in combination” refers to the use of more than one therapeutic agent. The use of the term “in combination” does not restrict the order in which the therapeutic agents are administered to a subject afflicted with cancer. A first therapeutic agent, such as a compound described herein, can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent or treatment, such as an anti-cancer agent, to a subject with prostate cancer.

As used herein, the terms “therapies” and “therapy” can refer to any protocol(s), method(s), and/or agent(s) that can be used in the prevention, treatment, management, or amelioration of cancer.

As used herein, a “protocol” includes dosing schedules and dosing regimens. The protocols herein are methods of use and include therapeutic protocols.

As used herein, the terms of “prostate cancer”, “metastatic prostate cancer”, “hormone-refractory prostate cancer”, “metastatic hormone-resistant prostate cancer”, “hormone independent prostate cancer”, “hormone dependent prostate cancer”, or “metastatic castration-resistant prostate cancer”, bear their normal meanings in the art. As is known, prostate cancer is the second leading cause of male cancer-related mortality in the United States. See, e.g., Jemal et al (2010), Cancer statistics, 2010. CA Cancer J Clin 60 277-300. A distinctive characteristic of this cancer type is that prostate tumors are critically dependent on androgen for development, growth and survival, with the transformative effects primarily mediated through activation of the androgen receptor (AR) signaling axis. See, e.g., Balk et al (2008), AR, the cell cycle, and prostate cancer. Nucl Recept Signal 6 e001; Chen et al (2008), Targeting the androgen receptor pathway in prostate cancer. Curr Opin Pharmacol 8 440-448; Li et al (2009), Mechanism of androgen receptor action. Maturitas 63 142-148. Androgen ablation therapy is the foundation of current prostate cancer treatment for patients that present with locally advanced or metastatic disease. This is typically achieved through chemical castration using selective antiandrogen agents, such as luteinizing hormone-releasing hormone (LHRH) agonists or newer AR inhibitors such as bicalutamide. See, e.g., Scher et al (2004), Targeting the androgen receptor: improving outcomes for castration-resistant prostate cancer. Endocr Relat Cancer 11 459-476. Although this approach initially induces clinical remissions, most patients ultimately relapse and progress to castration-resistant disease within a median of 18-24 months. See, e.g., Lonergan et al (2011), Androgen receptor signaling in prostate cancer development and progression. J Carcinog 10 20. While often termed ‘androgen-independent’ it is now clear that these tumors continue to rely on AR signaling, and a number of mechanisms have been proposed for reactivation of AR in the castrate environment. However, because of limited therapeutic options for advanced and metastatic prostate cancer, castrate-resistant tumors represent the lethal phase of the disease and patient prognosis is dismal. The incomplete efficacy of androgen deprivation therapy and lack of survival benefit from newer, targeted experimental approaches highlights an urgent need for novel treatment strategies to improve patient outcomes. See, e.g., Lassi et al (2010), Update on castrate-resistant prostate cancer: 2010. Curr Opin Oncol 22 263-267.

As used herein, one of the methods for the quantitative real-time PCR detection of serum level maspin is described here as for illustration only. Total RNA was extracted from the patient blood samples as described in Drachenberg et al, Circulating levels of interleukin-6 in patients with hormone refractory prostate cancer. Prostate, 1999; 41:127-33. The quality of the RNA was verified by agarose gel electrophoresis showing intact 185 and 28S rRNA, and by UV spectrophotometry showing an A₂₆₀ₙₘ/A₂₈₀ₙₘ ratio between 1.8 and 2. One microgram of each RNA sample was reverse-transcribed in a 20-mL reaction as described in the above-identified reference. For real-time PCR, 1 mL of the resulting cDNA was mixed with SYBR Green PCR Mastermix (Stratagene) (LaJolla, Calif.) and 300 nM of the PCR primers. The primers for maspin coding sequence were: 5′-CTACTTTGTTGGCAAGTGGATGAA-3′ and 5′-ACTGGTTTGGTGTCTGTCTTGTTG-3′. The primers for GAPDH were as previously described in the reference by Twillie et al, Interleukin-6: a candidate mediator of human prostate cancer morbidity. Urology, 1995; 45:542-9. The real-time PCR thermal profile was: 1 cycle of 95° C./10 min, 40 cycles of 95° C./30 sec--->55° C./1 min--->72° C./30 sec, 1 cycle of 95° C./1 min, and finally 41 cycles of 95° C.--->(55° C.+1° C./cycle)/30 sec. Critical threshold cycle numbers (Ct) were obtained using the built-in software of the Stratagene Mx4000™ Multiplex Quantitative PCR System. The measurement of maspin-specific cDNA species were normalized by the measurement of internal control GAPDH.

As used herein, a composition that “substantially” comprises a compound means that the composition contains more than about 80% by weight, more preferably more than about 90% by weight, even more preferably more than about 95% by weight, and most preferably more than about 97% by weight of the compound.

As used herein, the taxanes include paclitaxel (e.g., Taxal®), docetaxel and other paclitaxel analogs. Paclitaxel is an anti-cancer drug which can act by enhancing and stabilizing microtubule formation. Thus, the term “paclitaxel analog” is defined herein to mean a compound which has the basic paclitaxel skeleton and which stabilizes microtubule formation. Many analogs of paclitaxel are known, including docetaxel, also referred to as Taxotere®.

In addition, a paclitaxel analog can also be bonded to or be pendent from a pharmaceutically acceptable polymer, such as a polyacrylamide. The term “paclitaxel analog”, as it is used herein, includes such polymer linked taxanes.

The methods described herein can be understood more fully by reference to the following detailed description and illustrative examples, which are intended to exemplify non-limiting embodiments of the invention.

The methods described herein utilize compounds of Formulae (I) or (Ia) or those set forth in Table 1 or Table 2 and tautomers or pharmaceutically acceptable salts thereof.

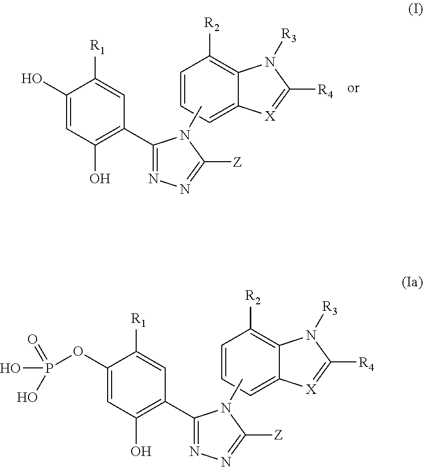

or a tautomer, or a pharmaceutically acceptable salt thereof, wherein:
Z is OH, SH, or NH₂;
X is CR₄ or N;
R₁ is —H, —OH, —SH, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanadino, a haloalkyl, a heteroalkyl, an alkoxy or cycloalkoxy, a haloalkoxy, —NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NR₈)NR₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)NR₁₀R₁₁, —SC(O)NR₁₀R₁₁, —SC(NR₈)NR₁₀R₁₁, —SC(S)NR₁₀R₁₁, —OC(NR₈)R₇, —SC(NR₈)R₇, —C(O)NR₁₀R₁₁, —NR₈C(O)R₇, —NR₇C(S)R₇, —NR₇C(S)OR₇, —NR₇C(NR₈)R₇, —NR₇C(O)OR₇, —NR₇C(NR₈)OR₇, —NR₇C(O)NR₁₀R₁₁, —NR₇C(S)NR₁₀R₁₁, —NR₇C(NR₈)NR₁₀R₁₁, —SR₇, —S(O)ₚR₇, —OS(O)ₚR₇, —OS(O)ₚOR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇, —SS(O)ₚNR₁₀R₁₁, —OP(O)(OR₇)₂, or —SP(O)(OR₇)₂;
R₂ is —H, —OH, —SH, —NR₇H, —OR₁₅, —SR₁₅, —NHR₁₅, —O(CH₂)ₘOH, —O(CH₂)SH, —O(CH₂)NR₇H, —S(CH₂)ₘOH, —S(CH₂)SH, —S(CH₂)NR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, or —NR₇C(NR₈)NR₁₀R₁₁;
R₃ is —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxyalkyl, alkoxyalkyl, a haloalkyl, a heteroalkyl, —C(O)R₇, —(CH₂)C(O)OR₇, —C(O)OR₇, —OC(O)R₇, —C(O)NR₁₀R₁₁, —S(O)ₚR₇, —S(O)ₚOR₇, or —S(O)ₚNR₁₀R₁₁;
R₄ is —H, —OH, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxyalkyl, alkoxyalkyl, halo, cyano, nitro, guanadino, a haloalkyl, a heteroalkyl, —C(O)R₇, —C(O)OR₇, —OC(O)R₇, —C(O)NR₁₀R₁₁, —NR₈C(O)R₇, —SR₇, —S(O)ₚR₇, —OS(O)ₚR₇, —S(O)ₚOR₇, —NR₈S(O)ₚR₇, —S(O)ₚNR₁₀R₁₁, or R₃ and R₄ taken together with the carbon atoms to which they are attached form an optionally substituted cycloalkenyl, an optionally substituted aryl, an optionally substituted heterocyclyl, or an optionally substituted heteroaryl;
R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;
R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl;
R₁₅, for each occurrence, is independently, a lower alkyl;
p, for each occurrence, is, independently, 1 or 2; and
m, for each occurrence, is independently, 1, 2, 3, or 4.

In one embodiment, in formula (I) or (Ia), X is CR₄. In another embodiment, in formula (I) or (Ia), X is N. In another embodiment, in formula (I) or (Ia), R₁ is selected from the group consisting of —H, lower alkyl, lower alkoxy, lower cycloalkyl, and lower cycloalkoxy. In another embodiment, in formula (I) or (Ia), R₁ is selected from the group consisting of —H, methyl, ethyl, propyl, isopropyl, cyclopropyl, methoxy, ethoxy, propoxy, and cyclopropoxy. In another embodiment, in formula (I) or (Ia), R₃ is selected from the group consisting of H, a lower alkyl, a lower cycloalkyl, —C(O)N(R₂₇)₂, and —C(O)OH, wherein R₂₇ is —H or a lower alkyl. In another embodiment, in formula (I) or (Ia), R₃ is selected from the group consisting of —H, methyl, ethyl, n-propyl, isopropyl, cyclopropyl, n-butyl, sec-butyl, tert-butyl, n-pentyl, n-hexyl, —C(O)OH, —(CH₂)ₘC(O)OH, —CH₂OCH₃, —CH₂CH₂OCH₃, and —C(O)N(CH₃)₂. In one embodiment, R₄ is H or a lower alkyl. In another embodiment, in formula (I) or (Ia), R₄ is selected from the group consisting of —H, methyl, ethyl, propyl, isopropyl or cyclopropyl. In another embodiment, in formula (I) or (Ia), R₁ is selected from the group consisting of —H, —OH, —SH, —NH₂, a lower alkoxy and a lower alkyl amino. In another embodiment, in formula (I) or (Ia), R₁ is selected from the group consisting of —H, —OH, methoxy and ethoxy. In another embodiment, in formula (I) or (Ia), Z is —OH. In another embodiment, in formula (I) or (Ia), Z is SH. In another embodiment, in formula (I) or (Ia), R₂ is selected from the group consisting of —H, —OH, —SH, —NH₂, a lower alkoxy and a lower alkyl amino. In another embodiment, in formula (I) or (Ia), R₂ is selected from the group consisting of —H, —OH, methoxy, and ethoxy. In another embodiment, in formula (I) or (Ia), R₁ is selected from the group consisting of —H, methyl, ethyl, propyl, isopropyl, cyclopropyl, methoxy, ethoxy, propoxy, and cyclopropoxy; R₃ is selected from the group consisting of —H, methyl, ethyl, n-propyl, isopropyl, cyclopropyl, n-butyl, sec-butyl, tert-butyl, n-pentyl, n-hexyl, —C(O)OH, —(CH₂)ₘC(O)OH, —CH₂OCH₃, —CH₂CH₂OCH₃, and —C(O)N(CH₃)₂; R₄ is selected from the group consisting of —H, methyl, ethyl, propyl, isopropyl or cyclopropyl; R₂ is selected from the group consisting of —H, —OH, —SH, —NH₂, a lower alkoxy and a lower alkyl amino; and Z is OH. In another embodiment, in formula (I) or (Ia), R₁ is selected from the group consisting of —H, methyl, ethyl, propyl, isopropyl, cyclopropyl, methoxy, ethoxy, propoxy, and cyclopropoxy; R₃ is selected from the group consisting of —H, methyl, ethyl, n-propyl, isopropyl, cyclopropyl, n-butyl, sec-butyl, tert-butyl, n-pentyl, n-hexyl, —C(O)OH, —(CH₂)ₘC(O)OH, —CH₂OCH₃, —CH₂CH₂OCH₃, and —C(O)N(CH₃)₂; R₄ is selected from the group consisting of —H, methyl, ethyl, propyl, isopropyl or cyclopropyl; R₂ is selected from the group consisting of —H, —OH, —SH, —NH₂, a lower alkoxy and a lower alkyl amino; and Z is SH.

In another embodiment, the compound is selected from the group consisting of: 3-(2,4-dihydroxy-5-ethyl-phenyl)-4-(1,3-dimethyl-indol-5-yl)-5-hydroxy-[1,2,4]triazole, dihydroxy-5-isopropyl-phenyl)-4-(1,3-dimethyl-indol-5-yl)-5-hydroxy-[1,2,4]triazole, 3-(2,4-dihydroxy-5-isopropyl-phenyl)-4-(1-methyl-indol-5-yl)-5-hydroxy-[1,2,4]triazole, dihydroxy-5-isopropyl-phenyl)-4-(1-isopropyl-indol-4-yl)-5-hydroxy-[1,2,4]triazole, dihydroxy-5-isopropyl-phenyl)-4-(1-methyl-indazol-5-yl)-5-mercapto-[1,2,4]triazole, dihydroxy-5-isopropyl-phenyl)-4-(1-methyl-indazol-6-yl)-5-mercapto-[1,2,4]triazole, dihydroxyphenyl)-4-(1-ethyl-indol-4-yl)-5-mercapto-[1,2,4]triazole, 3-(2,4-dihydroxyphenyl)-4-(1-isopropyl-indol-4-yl)-5-mercapto-[1,2,4]triazole, 3-(2,4-dihydroxyphenyl)-4-(indol-4-yl)-5-mercapto-[1,2,4]triazole, 3-(2,4-dihydroxyphenyl)-4-(1-methoxyethyl-indol-4-yl)-5-mercapto-[1,2,4]triazole, 3-(2,4-dihydroxy-5-ethyl-phenyl)-4-(1-isopropyl-indol-4-yl)-5-mercapto-[1,2,4]triazole, 3-(2,4-dihydroxyphenyl)-4-(1-dimethylcarbamoyl-indol-4-yl)-5-mercapto-[1,2,4]triazole, 3-(2,4-dihydroxy-5-ethyl-phenyl)-4-(1-propyl-indol-4-yl)-5-mercapto-[1,2,4]triazole, 3-(2,4-dihydroxy-5-ethyl-phenyl)-4-(1,2,3-trimethyl-indol-5-yl)-5-mercapto-[1,2,4]triazole, 3-(2,4-dihydroxy-5-ethyl-phenyl)-4-(2,3-dimethyl-indol-5-yl)-5-mercapto-[1,2,4]triazole, 3-(2,4-dihydroxy-5-ethyl-phenyl)-4-(1-acetyl-2,3-dimethyl-indol-5-yl)-5-mercapto-[1,2,4]triazole, 3-(2,4-dihydroxy-5-ethyl-phenyl)-4-(1-propyl-2,3-dimethyl-indol-5-yl)-5-mercapto-[1,2,4]triazole, 3-(2,4-dihydroxy-5-ethyl-phenyl)-4-(1-n-butyl-indol-4-yl)-5-mercapto-[1,2,4]triazole, 3-(2,4-dihydroxy-5-ethyl-phenyl)-4-(1-n-pentyl-indol-4-yl)-5-mercapto-[1,2,4]triazole, 3-(2,4-dihydroxy-5-ethyl-phenyl)-4-(1-n-hexyl-indol-4-yl)-5-mercapto-[1,2,4]triazole, 3-(2,4-dihydroxy-5-cyclopropyl-phenyl)-4-(1-(1-methylcyclopropyl)-indol-4-yl)-5-mercapto-[1,2,4]triazole, 3-(2,4-dihydroxy-5-cyclopropyl-phenyl)-4-(1,2,3-trimethyl-indol-5-yl)-5-mercapto-[1,2,4]triazole, 3-(2,4-dihydroxy-5-ethyl-phenyl)-4-(1-methyl-3-ethyl-indol-5-yl)-5-mercapto-[1,2,4]triazole, 3-(2,4-dihydroxy-5-ethyl-phenyl)-4-(1,3-dimethyl-indol-5-yl)-5-mercapto-[1,2,4]triazole, 3-(2,4-dihydroxy-5-ethyl-phenyl)-4-(1-methyl-3-isopropyl-indol-5-yl)-5-mercapto-[1,2,4]triazole, 3-(2,4-dihydroxy-5-ethyl-phenyl)-4-(1,2-dimethyl-indol-5-yl)-5-mercapto-[1,2,4]triazole, 3-(2,4-dihydroxy-5-ethyl-phenyl)-4-(N-methyl-indol-5-yl)-5-mercapto-[1,2,4]triazole, 3-(2,4-dihydroxy-5-isopropyl-phenyl)-4-(1,3-dimethyl-indol-5-yl)-5-mercapto-[1,2,4]triazole, 3-(2,4-dihydroxy-5-cyclopropyl-phenyl)-4-(1,3-dimethyl-indol-5-yl)-5-mercapto-[1,2,4]triazole, 3-(2,4-dihydroxy-5-cyclopropyl-phenyl)-4-(1-methyl-indol-5-yl)-5-mercapto-[1,2,4]triazole, 3-(2,4-dihydroxy-5-isopropyl-phenyl)-4-(1H-indol-5-yl)-5-mercapto-[1,2,4]triazole, 3-(2,4-dihydroxy-5-ethyl-phenyl)-4-(1,2-dimethyl-indol-5-yl)-5-mercapto-[1,2,4]triazole, 3-(2,4-dihydroxy-5-isopropyl-phenyl)-4-(1-ethyl-indol-5-yl)-5-mercapto-[1,2,4]triazole, 3-(2,4-dihydroxy-5-isopropyl-phenyl)-4-(1-propyl-indol-5-yl)-5-mercapto-[1,2,4]triazole, or a tautomer, or a pharmaceutically acceptable salt thereof.

In another embodiment, in formula (I) or (Ia), X is N.

In another embodiment, the compound is selected from the group consisting of 3-(2,4-dihydroxy-5-ethyl-phenyl)-4-(1-ethyl-benzimidazol-4-yl)-5-mercapto-[1,2,4]triazole, 3-(2,4-dihydroxy-5-ethyl-phenyl)-4-(1-ethyl-benzimidazol-4-yl)-5-mercapto-[1,2,4]triazole HCL salt, 3-(2,4-dihydroxy-5-ethyl-phenyl)-4-(2-methyl-3-ethyl-benzimidazol-5-yl)-5-mercapto-[1,2,4]triazole, 3-(2,4-dihydroxy-5-ethyl-phenyl)-4-(1-ethyl-2-methyl-benzimidazol-5-yl)-5-mercapto-[1,2,4]triazole, 3-(2,4-dihydroxy-5-isopropyl-phenyl)-4-(1-methyl-2-trifluoromethyl-benzimidazol-5-yl)-5-mercapto-[1,2,4]triazole, or a tautomer, or a pharmaceutically acceptable salt thereof.

i) Exemplary Triazolone Compounds

Exemplary compounds described herein are depicted in Table 1 below, including tautomers or pharmaceutically acceptable salts.

TABLE 1

| | Structure | Tautomeric Structure | Name |
|---|---|---|---|
| 1 | 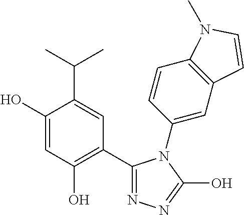 [SMILES: CC(C)c1cc(-c2nnc(O)n2-c2ccc3c(ccn3C)c2)c(O)cc1O] | 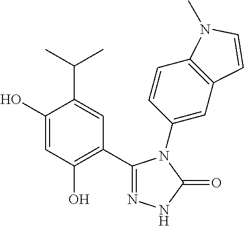 [SMILES: CC(C)c1cc(-c2n[nH]c(=O)n2-c2ccc3c(ccn3C)c2)c(O)cc1O] | 3-(2,4-dihydroxy-5- isopropyl-phenyl)-4-(1- methyl-indol-5-yl)-5- hydroxy-[1,2,4] triazole |
| 2 | 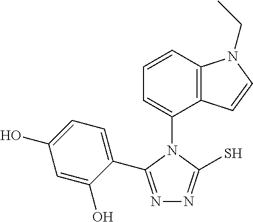 [SMILES: CCn1ccc2c(-n3c(S)nnc3-c3ccc(O)cc3O)cccc21] | 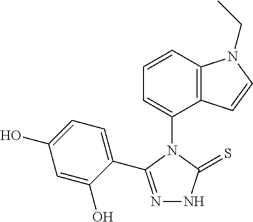 [SMILES: CCn1ccc2c(-n3c(-c4ccc(O)cc4O)n[nH]c3=S)cccc21] | 3-(2,4- Dihydroxyphenyl)-4-(1- ethyl-indol-4-yl)-5- mercapto-[1,2,4] triazole |
| 3 | 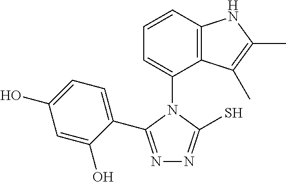 [SMILES: Cc1[nH]c2cccc(-n3c(S)nnc3-c3ccc(O)cc3O)c2c1C] | 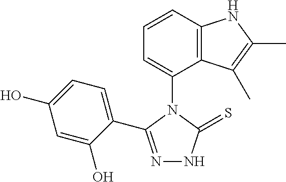 [SMILES: Cc1[nH]c2cccc(-n3c(-c4ccc(O)cc4O)n[nH]c3=S)c2c1C] | 3-(2,4-Dihydroxy- phenyl)-4-(2,3- dimethyl-1H-indol-4- yl)-5-mercapto-[1,2,4] triazole |
| 4 | 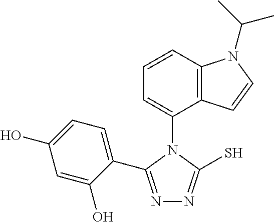 [SMILES: CC(C)n1ccc2c(-n3c(S)nnc3-c3ccc(O)cc3O)cccc21] | 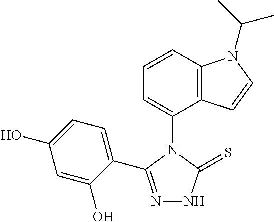 [SMILES: CC(C)n1ccc2c(-n3c(-c4ccc(O)cc4O)n[nH]c3=S)cccc21] | 3-(2,4- Dihydroxyphenyl)-4-(1- isopropyl-indol-4-yl)-5- mercapto-[1,2,4] triazole |
| 5 | 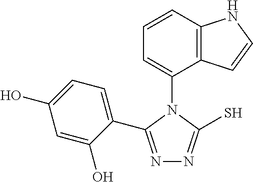 [SMILES: Oc1ccc(-c2nnc(S)n2-c2cccc3[nH]ccc23)c(O)c1] | 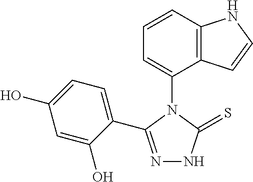 [SMILES: Oc1ccc(-c2n[nH]c(=S)n2-c2cccc3[nH]ccc23)c(O)c1] | 3-(2,4-Dihydroxy- phenyl)-4-(indol-4-yl)- 5-mercapto-[1,2,4] triazole |
| 6 | 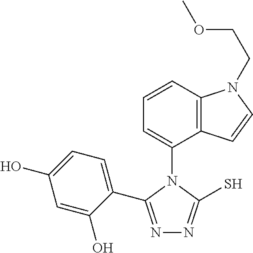 [SMILES: COCCn1ccc2c(-n3c(S)nnc3-c3ccc(O)cc3O)cccc21] | 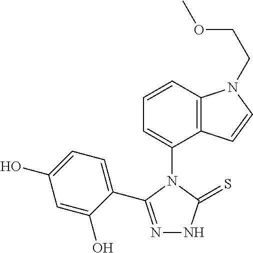 [SMILES: COCCn1ccc2c(-n3c(-c4ccc(O)cc4O)n[nH]c3=S)cccc21] | 3-(2,4-Dihydroxy- phenyl)-4-[1-(2- methoxyethoxy)-indol- 4-yl]-5-mercapto-[1,2,4] triazole |
| 7 | 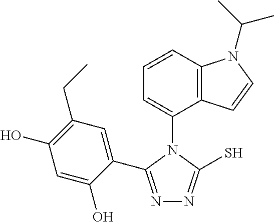 [SMILES: CCc1cc(-c2nnc(S)n2-c2cccc3c2ccn3C(C)C)c(O)cc1O] | 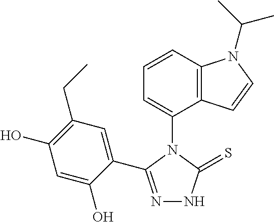 [SMILES: CCc1cc(-c2n[nH]c(=S)n2-c2cccc3c2ccn3C(C)C)c(O)cc1O] | 3-(2,4-Dihydroxy-5- ethyl-phenyl)-4-(1- isopropyl-indol-4-yl)-5- mercapto-[1,2,4] triazole |
| 8 | 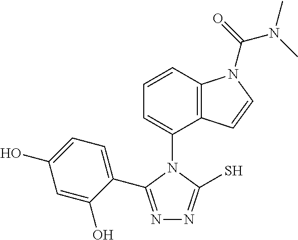 [SMILES: CN(C)C(=O)n1ccc2c(-n3c(S)nnc3-c3ccc(O)cc3O)cccc21] | 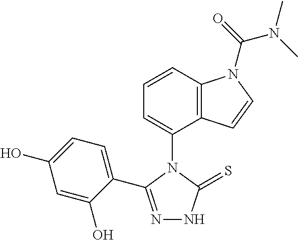 [SMILES: CN(C)C(=O)n1ccc2c(-n3c(-c4ccc(O)cc4O)n[nH]c3=S)cccc21] | 3-(2,4-Dihydroxy-5- ethyl-phenyl)-4-[1- (dimethyl-carbamoyl)- indol-4-yl]-5-mercapto- [1,2,4] triazole |
| 9 | 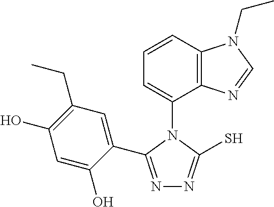 [SMILES: CCc1cc(-c2nnc(S)n2-c2cccc3c2ncn3CC)c(O)cc1O] | 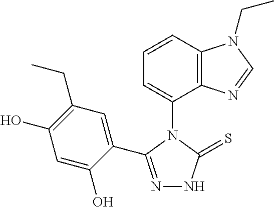 [SMILES: CCc1cc(-c2n[nH]c(=S)n2-c2cccc3c2ncn3CC)c(O)cc1O] | 3-(2,4-Dihydroxy-5- ethyl-phenyl)-4-(1- ethyl-benzoimidazol-4- yl)-5-mercapto-[1,2,4] triazole |
| 10 | 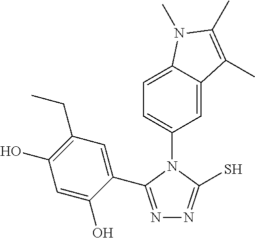 [SMILES: CCc1cc(-c2nnc(S)n2-c2ccc3c(c2)c(C)c(C)n3C)c(O)cc1O] | 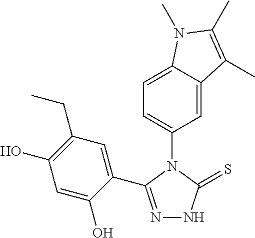 [SMILES: CCc1cc(-c2n[nH]c(=S)n2-c2ccc3c(c2)c(C)c(C)n3C)c(O)cc1O] | 3-(2,4-Dihydroxy-5- ethyl-phenyl)-4-(1,2,3- trimethyl-indol-5-yl)-5- mercapto-[1,2,4] triazole |
| 11 | 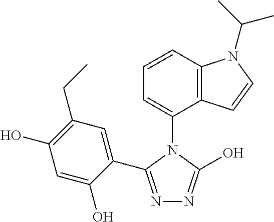 [SMILES: CCc1cc(-c2nnc(O)n2-c2cccc3c2ccn3C(C)C)c(O)cc1O] | 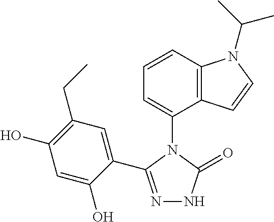 [SMILES: CCc1cc(-c2n[nH]c(=O)n2-c2cccc3c2ccn3C(C)C)c(O)cc1O] | 3-(2,4-Dihydroxy-5- ethyl-phenyl)-4-(1- isopropyl-indol-3-yl)-5- hydroxy-[1,2,4] triazole |
| 12 | 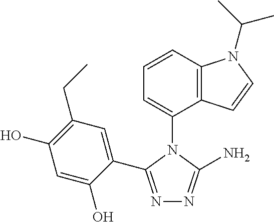 [SMILES: CCc1cc(-c2nnc(N)n2-c2cccc3c2ccn3C(C)C)c(O)cc1O] | 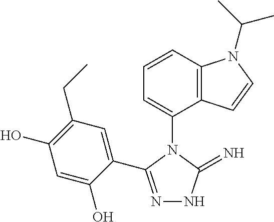 [SMILES: CCc1cc(-c2n[nH]c(=N)n2-c2cccc3c2ccn3C(C)C)c(O)cc1O] | 3-(2,4-Dihydroxy-5- ethyl-phenyl)-4-(1- isopropyl-indol-4-yl)-5- amino-[1,2,4] triazole |
| 15 | 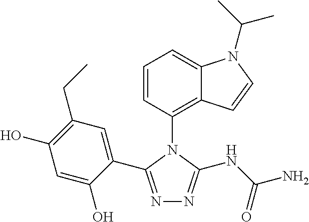 [SMILES: CCc1cc(-c2nnc(NC(N)=O)n2-c2cccc3c2ccn3C(C)C)c(O)cc1O] | | 3-(2,4-Dihydroxy-5- ethyl-phenyl)-4-(1- isopropyl-indol-4-yl)-5- ureido-[1,2,4] triazole |
| 16 | 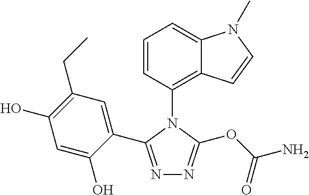 [SMILES: CCc1cc(-c2nnc(OC(N)=O)n2-c2cccc3c2ccn3C)c(O)cc1O] | | 3-(2,4-Dihydroxy-5- ethyl-phenyl)-4-(1- methyl-indol-4-yl)-5- carbamoyloxy-[1,2,4] triazole |
| 17 | 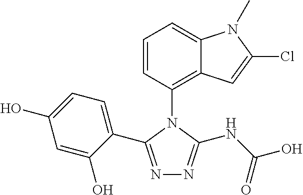 [SMILES: Cn1c(Cl)cc2c(-n3c(NC(=O)O)nnc3-c3ccc(O)cc3O)cccc21] | | 3-(2,4-Dihydroxy- phenyl)-4-(1-methyl-2- chloro-indol-4-yl)-5- carbamoyloxy-[1,2,4] triazole |
| 18 | 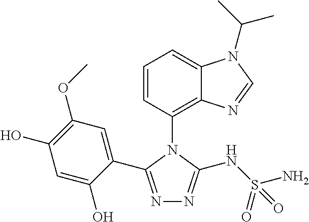 [SMILES: COc1cc(-c2nnc(NS(N)(=O)=O)n2-c2cccc3c2ncn3C(C)C)c(O)cc1O] | | 3-(2,4-Dihydroxy-5- methoxy-phenyl)-4-(1- isopropyl- benzoimidazol-4-yl)-5- (sulfamoylamino)- [1,2,4] triazole |
| 20 | 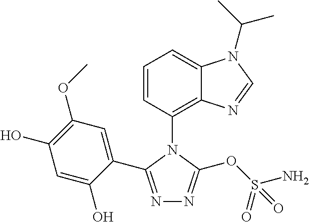 [SMILES: COc1cc(-c2nnc(OS(N)(=O)=O)n2-c2cccc3c2ncn3C(C)C)c(O)cc1O] | | 3-(2,4-Dihydroxy-5- methoxy-phenyl)-4-(1- isopropyl- benzoimidazol-4-yl)-5- (sulfamoyloxy)-[1,2,4] triazole |
| 21 | 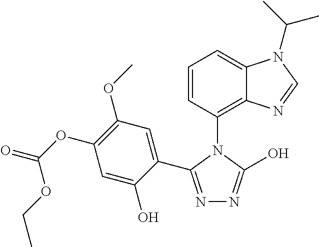 [SMILES: CCOC(=O)Oc1cc(O)c(-c2nnc(O)n2-c2cccc3c2ncn3C(C)C)cc1OC] | 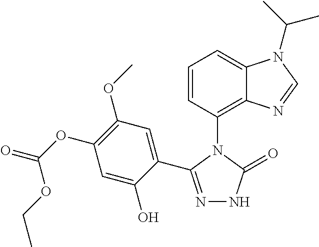 [SMILES: CCOC(=O)Oc1cc(O)c(-c2n[nH]c(=O)n2-c2cccc3c2ncn3C(C)C)cc1OC] | 3-(2-Hydroxy-4- ethoxycarbonyoxy-5- methoxy-phenyl)-4-(1- isopropyl- benzoimidazol-4-yl)-5- hydroxy-[1,2,4] triazole |
| 22 | 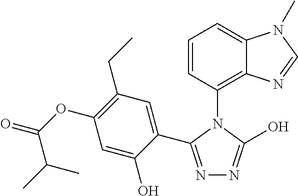 [SMILES: CCc1cc(-c2nnc(O)n2-c2cccc3c2ncn3C)c(O)cc1OC(=O)C(C)C] | 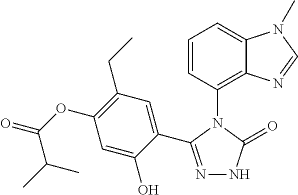 [SMILES: CCc1cc(-c2n[nH]c(=O)n2-c2cccc3c2ncn3C)c(O)cc1OC(=O)C(C)C] | 3-[2-Hydroxy-4- isobutyryloxy-5-ethyl- phenyl]-4-(1-methyl- benzo-imidazol-4-yl)-5- hydroxy-[1,2,4] triazole |
| 23 | 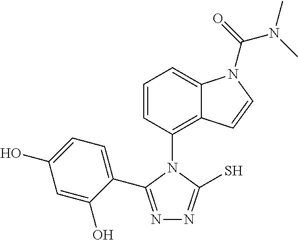 [SMILES: CN(C)C(=O)n1ccc2c(-n3c(S)nnc3-c3ccc(O)cc3O)cccc21] | 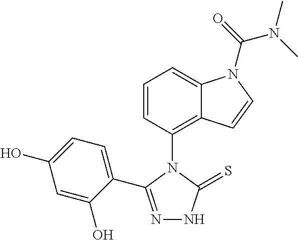 [SMILES: CN(C)C(=O)n1ccc2c(-n3c(-c4ccc(O)cc4O)n[nH]c3=S)cccc21] | 3-(2,4-Dihydroxy- phenyl)-4-(1- dimethylcarbamoyl- indol-4-yl)-5-mercapto- [1,2,4] triazole |
| 24 | 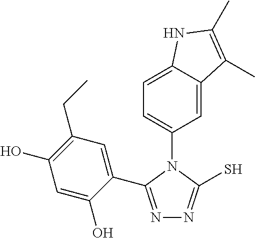 [SMILES: CCc1cc(-c2nnc(S)n2-c2ccc3[nH]c(C)c(C)c3c2)c(O)cc1O] | 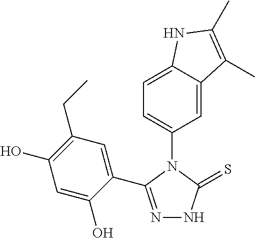 [SMILES: CCc1cc(-c2n[nH]c(=S)n2-c2ccc3[nH]c(C)c(C)c3c2)c(O)cc1O] | 3-(2,4-Dihydroxy-5- ethyl-phenyl)-4-(2,3- dimethyl-indol-5-yl)-5- mercapto-[1,2,4] triazole |
| 25 | 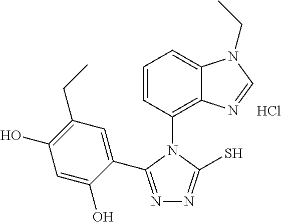 [SMILES: CCc1cc(-c2nnc(S)n2-c2cccc3c2ncn3CC)c(O)cc1O ; Cl] | 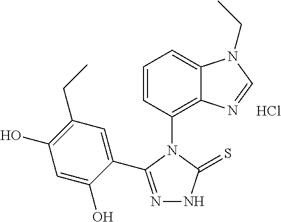 [SMILES: CCc1cc(-c2n[nH]c(=S)n2-c2cccc3c2ncn3CC)c(O)cc1O ; Cl] | 3-(2,4-Dihydroxy-5- ethyl-phenyl)-4-(1- ethyl-1H- benzoimidazol-4-yl)-5- mercapto-[1,2,4] triazole, HCl salt |
| 26 | 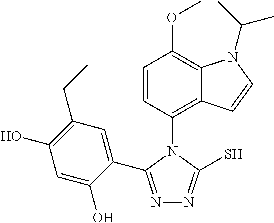 [SMILES: CCc1cc(-c2nnc(S)n2-c2ccc(OC)c3c2ccn3C(C)C)c(O)cc1O] | 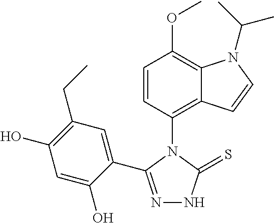 [SMILES: CCc1cc(-c2n[nH]c(=S)n2-c2ccc(OC)c3c2ccn3C(C)C)c(O)cc1O] | 3-(2,4-Dihydroxy-5- ethyl-phenyl)-4-(1- isopropyl-7-methoxy- indol-4-yl)-5-mercapto- [1,2,4] triazole |
| 27 | 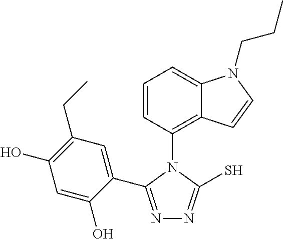 [SMILES: CCCn1ccc2c(-n3c(S)nnc3-c3cc(CC)c(O)cc3O)cccc21] | 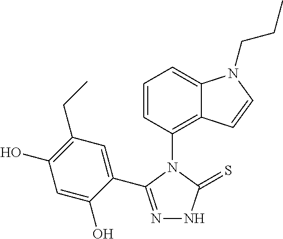 [SMILES: CCCn1ccc2c(-n3c(-c4cc(CC)c(O)cc4O)n[nH]c3=S)cccc21] | 3-(2,4-dihydroxy-5- ethyl-phenyl)-4-(1- propyl-indol-4-yl)-5- mercapto-[1,2,4] triazole |
| 28 | 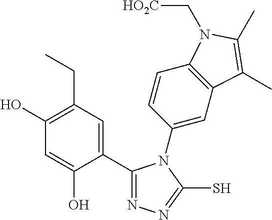 [SMILES: CCc1cc(-c2nnc(S)n2-c2ccc3c(c2)c(C)c(C)n3CC(=O)O)c(O)cc1O] | 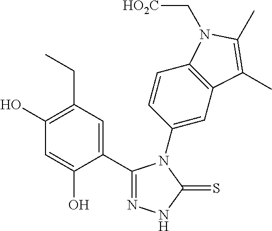 [SMILES: CCc1cc(-c2n[nH]c(=S)n2-c2ccc3c(c2)c(C)c(C)n3CC(=O)O)c(O)cc1O] | 3-(2,4-dihydroxy-5- ethyl-phenyl)-4-(1- acetyl-2,3-dimethyl- indol-5-yl)-5-mercapto- [1,2,4] triazole |
| 29 | 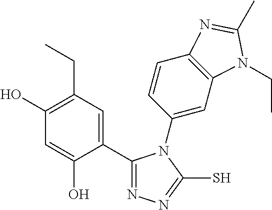 [SMILES: CCc1cc(-c2nnc(S)n2-c2ccc3nc(C)n(CC)c3c2)c(O)cc1O] | 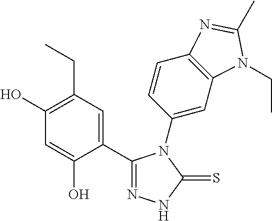 [SMILES: CCc1cc(-c2n[nH]c(=S)n2-c2ccc3nc(C)n(CC)c3c2)c(O)cc1O] | 3-(2,4-dihydroxy-5- ethyl-phenyl)-4-(2- methyl-3-ethyl- benzimidazol-5-yl)-5- mercapto-[1,2,4] triazole |
| 30 | 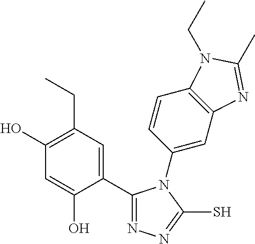 [SMILES: CCc1cc(-c2nnc(S)n2-c2ccc3c(c2)nc(C)n3CC)c(O)cc1O] | 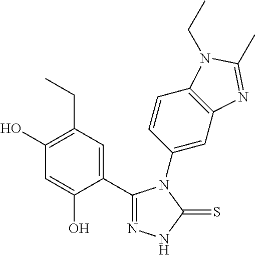 [SMILES: CCc1cc(-c2n[nH]c(=S)n2-c2ccc3c(c2)nc(C)n3CC)c(O)cc1O] | 3-(2,4-dihydroxy-5- ethyl-phenyl)-4-(1- ethyl-2-methyl- benzimidazol-5-yl)-5- mercapto-[1,2,4] triazole |
| 31 | 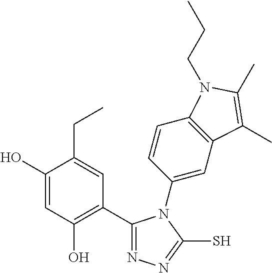 [SMILES: CCCn1c(C)c(C)c2cc(-n3c(S)nnc3-c3cc(CC)c(O)cc3O)ccc21] | 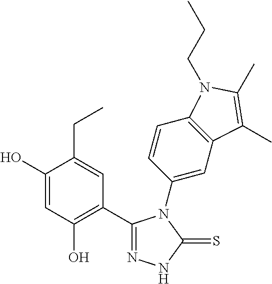 [SMILES: CCCn1c(C)c(C)c2cc(-n3c(-c4cc(CC)c(O)cc4O)n[nH]c3=S)ccc21] | 3-(2,4-dihydroxy-5- ethyl-phenyl)-4-(1- propyl-2,3-dimethyl- indol-5-yl)-5-mercapto- [1,2,4] triazole |
| 34 | 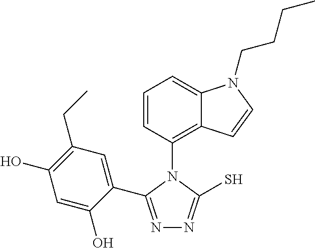 [SMILES: CCCCn1ccc2c(-n3c(S)nnc3-c3cc(CC)c(O)cc3O)cccc21] | 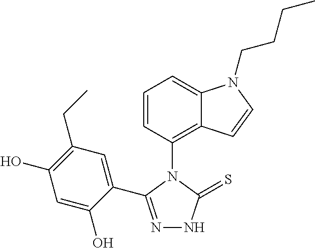 [SMILES: CCCCn1ccc2c(-n3c(-c4cc(CC)c(O)cc4O)n[nH]c3=S)cccc21] | 3-(2,4-dihydroxy-5- ethyl-phenyl)-4-(1-n- butyl-indol-4-yl)-5- mercapto-[1,2,4] triazole |
| 35 | 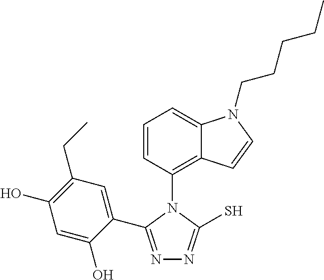 [SMILES: CCCCCn1ccc2c(-n3c(S)nnc3-c3cc(CC)c(O)cc3O)cccc21] | 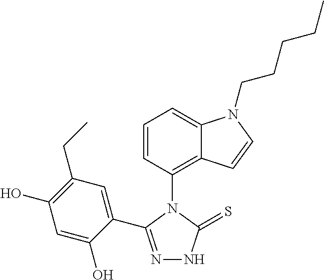 [SMILES: CCCCCn1ccc2c(-n3c(-c4cc(CC)c(O)cc4O)n[nH]c3=S)cccc21] | 3-(2,4-dihydroxy-5- ethyl-phenyl)-4-(1-n- pentyl-indol-4-yl)-5- mercapto-[1,2,4] triazole |
| 36 | 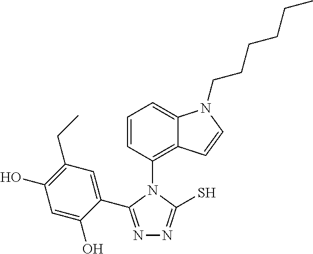 [SMILES: CCCCCCn1ccc2c(-n3c(S)nnc3-c3cc(CC)c(O)cc3O)cccc21] | 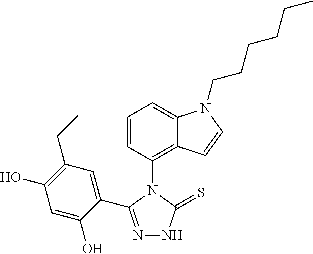 [SMILES: CCCCCCn1ccc2c(-n3c(-c4cc(CC)c(O)cc4O)n[nH]c3=S)cccc21] | 3-(2,4-dihydroxy-5- ethyl-phenyl)-4-(1-n- hexyl-indol-4-yl)-5- mercapto-[1,2,4] triazole |
| 37 | 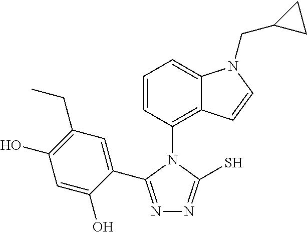 [SMILES: CCc1cc(-c2nnc(S)n2-c2cccc3c2ccn3CC2CC2)c(O)cc1O] | 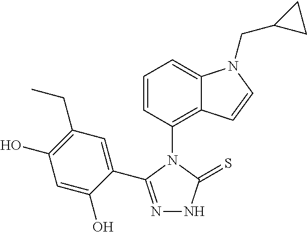 [SMILES: CCc1cc(-c2n[nH]c(=S)n2-c2cccc3c2ccn3CC2CC2)c(O)cc1O] | 3-(2,4-dihydroxy-5- cyclopropyl-phenyl)-4- (1-(1- methylcyclopropyl)- indol-4-yl)-5-mercapto- [1,2,4] triazole |
| 38 | 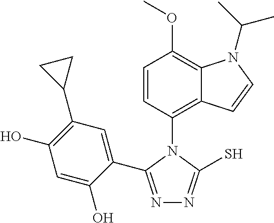 [SMILES: COc1ccc(-n2c(S)nnc2-c2cc(C3CC3)c(O)cc2O)c2ccn(C(C)C)c12] | 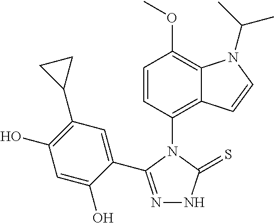 [SMILES: COc1ccc(-n2c(-c3cc(C4CC4)c(O)cc3O)n[nH]c2=S)c2ccn(C(C)C)c12] | 3-(2,4-dihydroxy-5- cyclopropyl-phenyl)-4- (1-isopropyl-7- methoxy-indol-4-yl)-5- mercapto-[1,2,4] triazole |
| 39 | 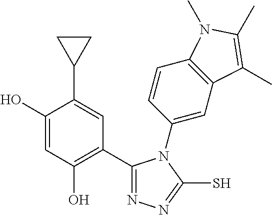 [SMILES: Cc1c(C)n(C)c2ccc(-n3c(S)nnc3-c3cc(C4CC4)c(O)cc3O)cc12] | 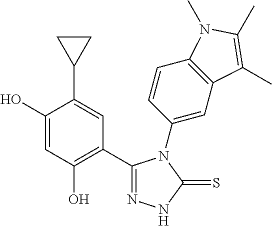 [SMILES: Cc1c(C)n(C)c2ccc(-n3c(-c4cc(C5CC5)c(O)cc4O)n[nH]c3=S)cc12] | 3-(2,4-dihydroxy-5- cyclopropyl-phenyl)-4- (1,2,3-trimethyl-indol-5- yl)-5-mercapto-[1,2,4] triazole |
| 40 | 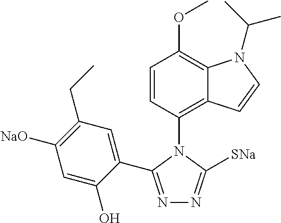 | | 3-(2,4-dihydroxy-5- ethyl-phenyl)-4-(1- isopropyl-7-methoxy- indol-4-yl)-5-mercapto- [1,2,4] triazole disodium salt |
| 41 | 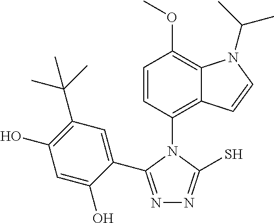 [SMILES: COc1ccc(-n2c(S)nnc2-c2cc(C(C)(C)C)c(O)cc2O)c2ccn(C(C)C)c12] | 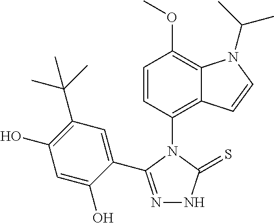 [SMILES: COc1ccc(-n2c(-c3cc(C(C)(C)C)c(O)cc3O)n[nH]c2=S)c2ccn(C(C)C)c12] | 3-(2,4-dihydroxy-5-tert- butyl-phenyl)-4-(1- isopropyl-7-methoxy- indol-4-yl)-5-mercapto- [1,2,4] triazole |
| 42 | 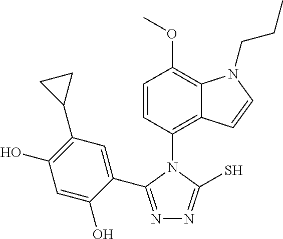 [SMILES: CCCn1ccc2c(-n3c(S)nnc3-c3cc(C4CC4)c(O)cc3O)ccc(OC)c21] | 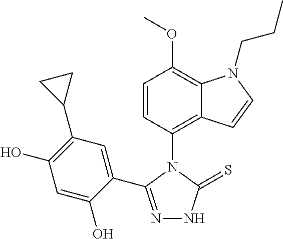 [SMILES: CCCn1ccc2c(-n3c(-c4cc(C5CC5)c(O)cc4O)n[nH]c3=S)ccc(OC)c21] | 3-(2,4-dihydroxy-5- cyclopropyl-phenyl)-4- (1-propyl-7-methoxy- indol-4-yl)-5-mercapto- [1,2,4] triazole |
| 43 | 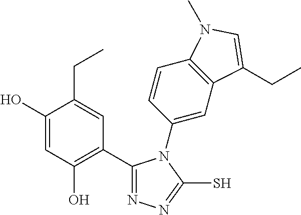 [SMILES: CCc1cc(-c2nnc(S)n2-c2ccc3c(c2)c(CC)cn3C)c(O)cc1O] | 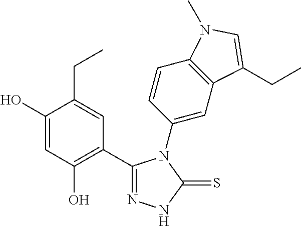 | 3-(2,4-dihydroxy-5- ethyl-phenyl)-4-(1- methyl-3-ethyl-indol-5- yl)-5-mercapto-[1,2,4] triazole |
| 44 | 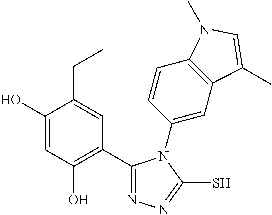 [SMILES: CCc1cc(-c2nnc(S)n2-c2ccc3c(c2)c(C)cn3C)c(O)cc1O] | 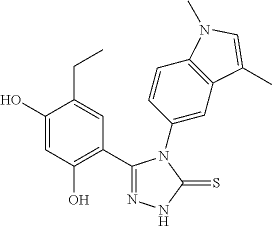 | 3-(2,4-dihydroxy-5- ethyl-phenyl)-4-(1,3- dimethyl-indol-5-yl)-5- mercapto-[1,2,4] triazole |
| 45 | 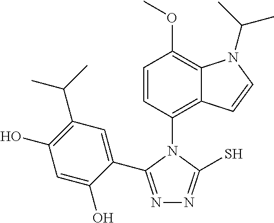 [SMILES: COc1ccc(-n2c(S)nnc2-c2cc(C(C)C)c(O)cc2O)c2ccn(C(C)C)c12] | 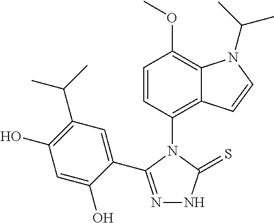 [SMILES: COc1ccc(-n2c(-c3cc(C(C)C)c(O)cc3O)n[nH]c2=S)c2ccn(C(C)C)c12] | 3-(2,4-dihydroxy-5- isopropyl-phenyl)-4-(1- isopropyl-7-methoxy- indol-4-yl)-5-mercapto- [1,2,4] triazole |
| 46 | 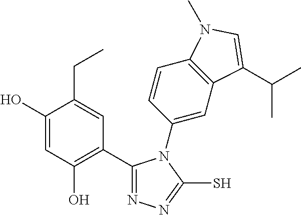 [SMILES: CCc1cc(-c2nnc(S)n2-c2ccc3c(c2)c(C(C)C)cn3C)c(O)cc1O] | 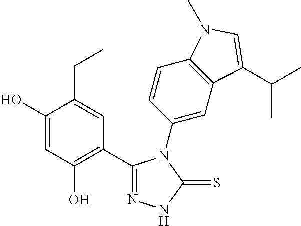 | 3-(2,4-dihydroxy-5- ethyl-phenyl)-4-(1- methyl-3-isopropyl- indol-5-yl)-5-mercapto- [1,2,4] triazole |
| 48 | 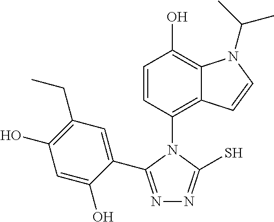 [SMILES: CCc1cc(-c2nnc(S)n2-c2ccc(O)c3c2ccn3C(C)C)c(O)cc1O] | 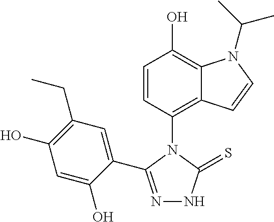 [SMILES: CCc1cc(-c2n[nH]c(=S)n2-c2ccc(O)c3c2ccn3C(C)C)c(O)cc1O] | 3-(2,4-dihydroxy-5- ethyl-phenyl)-4-(1- isopropyl-7-hydroxy- indol-4-yl)-5-mercapto- [1,2,4] triazole |
| 49 | 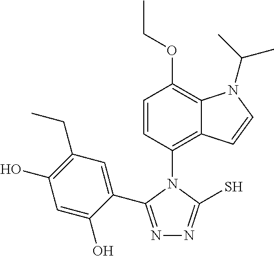 [SMILES: CCOc1ccc(-n2c(S)nnc2-c2cc(CC)c(O)cc2O)c2ccn(C(C)C)c12] | 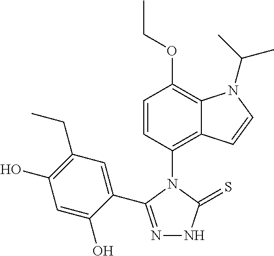 [SMILES: CCOc1ccc(-n2c(-c3cc(CC)c(O)cc3O)n[nH]c2=S)c2ccn(C(C)C)c12] | 3-(2,4-dihydroxy-5- ethyl-phenyl)-4-(1- isopropyl-7-ethoxy- indol-4-yl)-5-mercapto- [1,2,4] triazole |
| 50 | 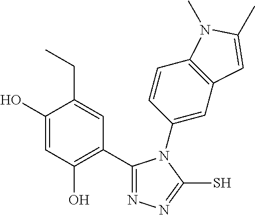 [SMILES: CCc1cc(-c2nnc(S)n2-c2ccc3c(c2)cc(C)n3C)c(O)cc1O] | 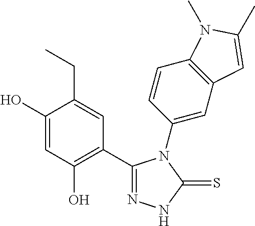 | 3-(2,4-dihydroxy-5- ethyl-phenyl)-4-(1,2- dimethyl-indol-5-yl)-5- mercapto-[1,2,4] triazole |
| 51 | 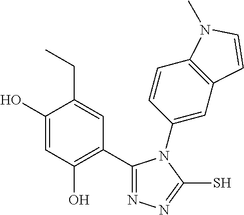 [SMILES: CCc1cc(-c2nnc(S)n2-c2ccc3c(ccn3C)c2)c(O)cc1O] | 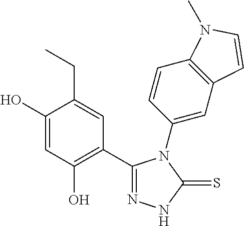 | 3-(2,4-dihydroxy-5- ethyl-phenyl)-4-(N- methyl-indol-5-yl)-5- mercapto-[1,2,4] triazole |
| 55 | 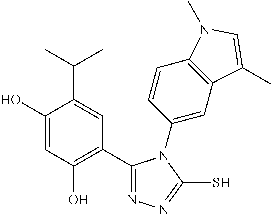 [SMILES: Cc1cn(C)c2ccc(-n3c(S)nnc3-c3cc(C(C)C)c(O)cc3O)cc12] | 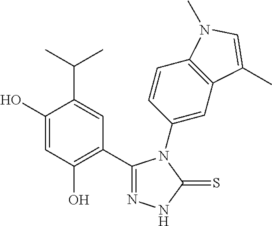 | 3-(2,4-dihydroxy-5- isopropyl-phenyl)-4- (1,3-dimethyl-indol-5- yl)-5-mercapto-[1,2,4] triazole |
| 56 | 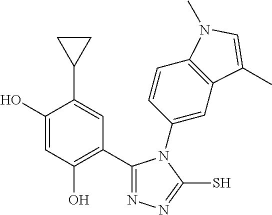 [SMILES: Cc1cn(C)c2ccc(-n3c(S)nnc3-c3cc(C4CC4)c(O)cc3O)cc12] | 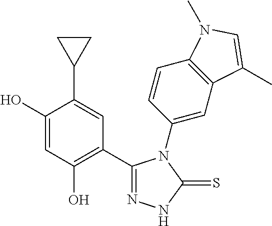 | 3-(2,4-dihydroxy-5- cyclopropyl-phenyl)-4- (1,3-dimethyl-indol-5- yl)-5-mercapto-[1,2,4] triazole |
| 57 | 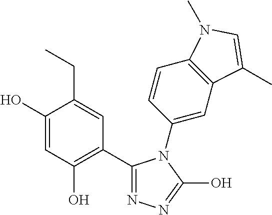 [SMILES: CCc1cc(-c2nnc(O)n2-c2ccc3c(c2)c(C)cn3C)c(O)cc1O] | 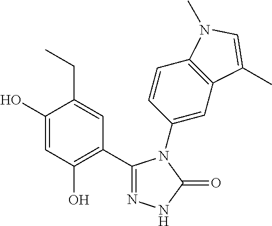 | 3-(2,4-dihydroxy-5- ethyl-phenyl)-4-(1,3- dimethyl-indol-5-yl)-5- hydroxy-[1,2,4] triazole |
| 58 | 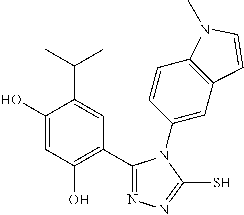 [SMILES: CC(C)c1cc(-c2nnc(S)n2-c2ccc3c(ccn3C)c2)c(O)cc1O] | 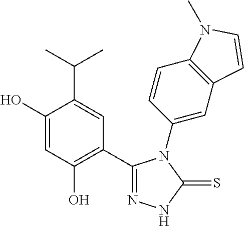 [SMILES: CC(C)c1cc(-c2n[nH]c(=S)n2-c2ccc3c(ccn3C)c2)c(O)cc1O] | 3-(2,4-dihydroxy-5- isopropyl-phenyl)-4-(N- methyl-indol-5-yl)-5- mercapto-[1,2,4] triazole |
| 59 | 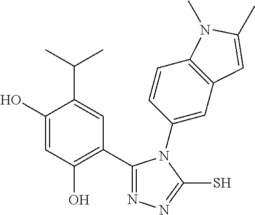 [SMILES: Cc1cc2cc(-n3c(S)nnc3-c3cc(C(C)C)c(O)cc3O)ccc2n1C] | 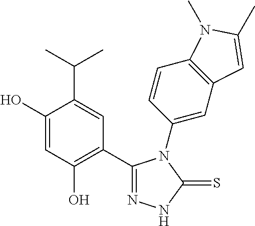 [SMILES: Cc1cc2cc(-n3c(-c4cc(C(C)C)c(O)cc4O)n[nH]c3=S)ccc2n1C] | 3-(2,4-dihydroxy-5- isopropyl-phenyl)-4- (1,2-dimethyl-indol-5- yl)-5-mercapto-[1,2,4] triazole |
| 60 | 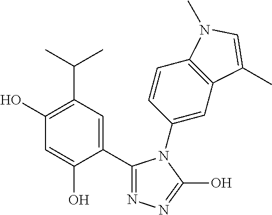 [SMILES: Cc1cn(C)c2ccc(-n3c(O)nnc3-c3cc(C(C)C)c(O)cc3O)cc12] | 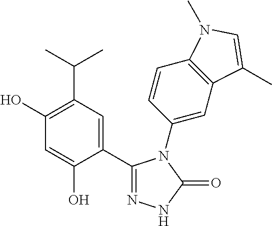 [SMILES: Cc1cn(C)c2ccc(-n3c(-c4cc(C(C)C)c(O)cc4O)n[nH]c3=O)cc12] | 3-(2,4-dihydroxy-5- isopropyl-phenyl)-4 - (1,3-dimethyl-indol-5- yl)-5-hydroxy-[1,2,4] triazole |
| 62 | 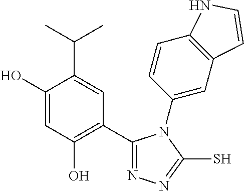 [SMILES: CC(C)c1cc(-c2nnc(S)n2-c2ccc3[nH]ccc3c2)c(O)cc1O] | 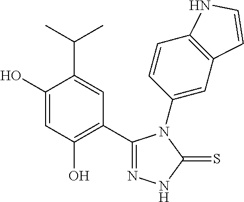 [SMILES: CC(C)c1cc(-c2n[nH]c(=S)n2-c2ccc3[nH]ccc3c2)c(O)cc1O] | 3-(2,4-dihydroxy-5- isopropyl-phenyl)-4- (1H-indol-5-yl)-5- mercapto-[1,2,4] triazole |
| 63 | 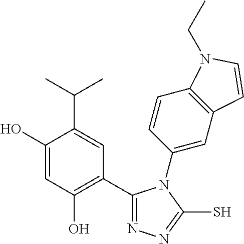 [SMILES: CCn1ccc2cc(-n3c(S)nnc3-c3cc(C(C)C)c(O)cc3O)ccc21] | 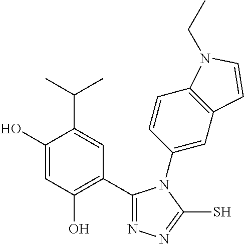 [SMILES: CCn1ccc2cc(-n3c(S)nnc3-c3cc(C(C)C)c(O)cc3O)ccc21] | 3-(2,4-dihydroxy-5- isopropyl-phenyl)-4-(1- ethyl-indol-5-yl)-5- mercapto-[1,2,4] triazole |
| 64 | 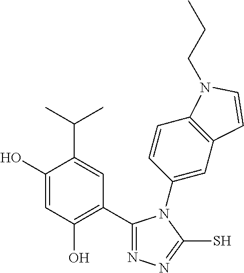 [SMILES: CCCn1ccc2cc(-n3c(S)nnc3-c3cc(C(C)C)c(O)cc3O)ccc21] | 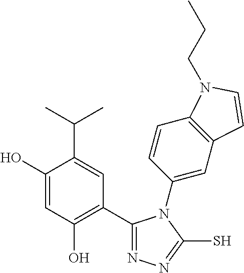 [SMILES: CCCn1ccc2cc(-n3c(S)nnc3-c3cc(C(C)C)c(O)cc3O)ccc21] | 3-(2,4-dihydroxy-5- isopropyl-phenyl)-4-(1- propyl-indol-5-yl)-5- mercapto-[1,2,4] triazole |
| 65 | 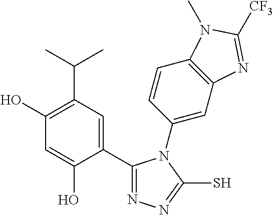 [SMILES: CC(C)c1cc(-c2nnc(S)n2-c2ccc3c(c2)nc(C(F)(F)F)n3C)c(O)cc1O] | 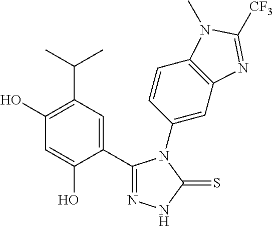 [SMILES: CC(C)c1cc(-c2n[nH]c(=S)n2-c2ccc3c(c2)nc(C(F)(F)F)n3C)c(O)cc1O] | 3-(2,4-dihydroxy-5- isopropyl-phenyl)-4-(1- methyl-2- trifluoromethyl- benzimidazol-5-yl)-5- mercapto-[1,2,4] triazole |
| 66 | 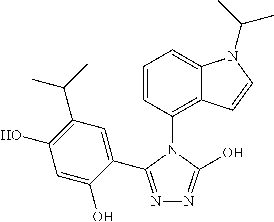 [SMILES: CC(C)c1cc(-c2nnc(O)n2-c2cccc3c2ccn3C(C)C)c(O)cc1O] | 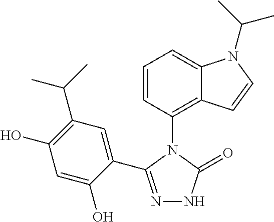 [SMILES: CC(C)c1cc(-c2n[nH]c(=O)n2-c2cccc3c2ccn3C(C)C)c(O)cc1O] | 3-(2,4-dihydroxy-5- isopropyl-phenyl)-4-(1- isopropyl-indol-4-yl)-5- hydroxy-[1,2,4] triazole |

TABLE 2
Compounds according to Formula (Ia)

| No. | Structure | Tautomeric structure | Name |
|---|---|---|---|
| 1a | 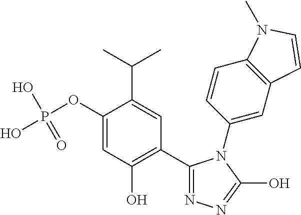 [SMILES: CC(C)c1cc(-c2nnc(O)n2-c2ccc3c(ccn3C)c2)c(O)cc1OP(=O)(O)O] | 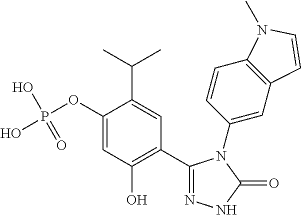 [SMILES: CC(C)c1cc(-c2n[nH]c(=O)n2-c2ccc3c(ccn3C)c2)c(O)cc1OP(=O)(O)O] | 5-hydroxy-4-(5- hydroxy-4-(1- methyl-1H-indol- 5-yl)-4H-1,2,4 - triazol-3-yl)-2- isopropylphenyl dihydrogen phosphate |
| 2a | 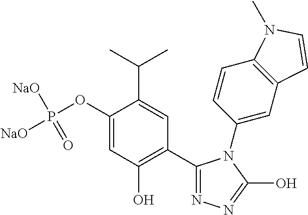 | 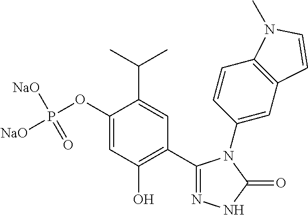 | sodium 5- hydroxy-4-(5- hydroxy-4-(1- methyl-1H-indol- 5-yl)-4H-1,2,4- triazol-3-yl)-2- isopropylphenyl phosphate |
| 3a | 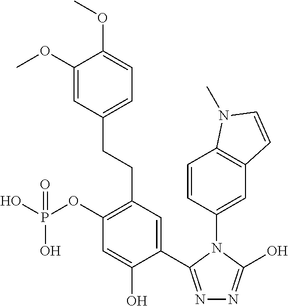 [SMILES: COc1ccc(CCc2cc(-c3nnc(O)n3-c3ccc4c(ccn4C)c3)c(O)cc2OP(=O)(O)O)cc1OC] | 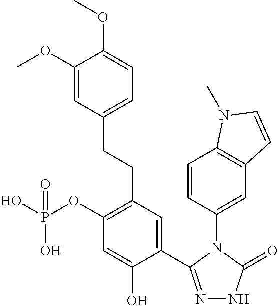 [SMILES: COc1ccc(CCc2cc(-c3n[nH]c(=O)n3-c3ccc4c(ccn4C)c3)c(O)cc2OP(=O)(O)O)cc1OC] | 2-(3,4- dimethoxyphenethyl)- 5-hydroxy- 4-(5-hydroxy-4- (1-methyl-1H- indol-5-yl)-4H- 1,2,4-triazol-3- yl)phenyl dihydrogen phosphate |
| 4a | 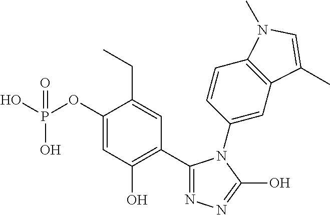 [SMILES: CCc1cc(-c2nnc(O)n2-c2ccc3c(c2)c(C)cn3C)c(O)cc1OP(=O)(O)O] | 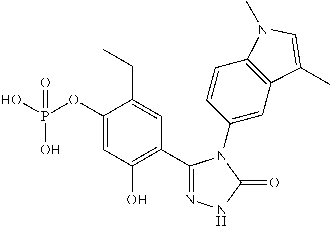 [SMILES: CCc1cc(-c2n[nH]c(=O)n2-c2ccc3c(c2)c(C)cn3C)c(O)cc1OP(=O)(O)O] | 4-(4-(1,3- dimethyl-1H- indol-5-yl)-5- hydroxy-4H- 1,2,4-triazol-3- yl)-2-ethyl-5- hydroxyphenyl dihydrogen phosphate |

Compounds used in the disclosed methods can be prepared according to methods disclosed in U.S. Publication No. 2006-0167070, and WO2009/023211.

Compounds described herein typically can form a tautomeric structure as shown below and as exemplified by the tautomeric structures shown in Tables 1 and 2:

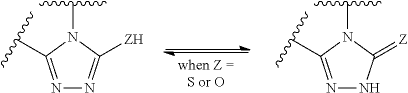

These triazolone Hsp90 inhibitors are particularly effective in treating patients with prostate cancer, such as metastatic prostate cancer, metastatic hormone-resistant prostate cancer or metastatic castration-resistant prostate cancer, or prostate cancer wherein it was previously treated with docetaxel-based chemotherapy.

The present method utilizes Hsp90 inhibitory compounds of formula (I) or (Ia) or compounds in Tables 1 or 2, or pharmaceutically acceptable salts or tautomers thereof, for the treatment of prostate cancer.

In one embodiment, the method includes the step of administering to a subject with prostate cancer an amount of from about 2 mg/m² to about 500 mg/m² of the triazolone compound of formula (I) or (Ia) or a compound in Tables 1 or 2. In one embodiment, the triazolone compound administered is from about 2 mg/m² to about 260 mg/m². In one embodiment, the compound of formula (I) or (Ia) or in Tables 1 or 2 may be administered weekly. In one embodiment, the compound of formula (I) or (Ia) or in Tables 1 or 2 may be administered twice weekly. In one embodiment, the compound may be administered for about 3 weeks. In another embodiment, the twice weekly administration for 3 weeks may be repeated after about 7 days dose-free. In one embodiment, the twice weekly administration after 7 days dose free may be repeated two or more times. In any one of these embodiments, the triazolone compound may be 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof. In any one of these embodiments, the triazolone compound may be 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof. In any one of these embodiments, the prostate cancer may be metastatic hormone-resistant prostate cancer. In any one of these embodiments, the prostate cancer may be metastatic castration-resistant prostate cancer. In any one of these embodiments, the prostate cancer may be previously treated with docetaxel-based chemotherapy.

In one embodiment, the amount of the compound of formula (I) or (Ia) or in Tables 1 or 2 administered is from about 2 mg/m² to about 500 mg/m², for example, from about 100 mg/m² to about 500 mg/m², from about 125 mg/m² to about 500 mg/m², from about 150 mg/m² to about 500 mg/m² or from about 175 mg/m² to about 500 mg/m², or from about 2 mg/m² to about 260 mg/m². In one embodiment, the amount of the compound of formula (I) or (Ia) or in Tables 1 or 2 administered is about 100 mg/m² to about 300 mg/m², from about 125 mg/m² to about 300 mg/m², from about 150 mg/m² to about 300 mg/m² or from about 175 mg/m² to about 300 mg/m². In some embodiments, the amount of the compound of formula (I) or (Ia) or in Tables 1 or 2 administered is about 2 mg/m², 4 mg/m², about 7 mg/m², about 10 mg/m², about 14 mg/m², about 19 mg/m², about 23 mg/m², about 25 mg/m², about 33 mg/m², about 35 mg/m², about 40 mg/m², about 48 mg/m², about 49 mg/m², about 50 mg/m², about 65 mg/m², about 75 mg/m², about 85 mg/m², about 100 mg/m², about 110 mg/m², about 115 mg/m², about 120 mg/m², about 145 mg/m², about 150 mg/m², about 175 mg/m², about 180 mg/m², about 215 mg/m² or about 260 mg/m².

The language “twice weekly” includes administration of a compound of formula (I) or (Ia) or in Tables 1 or 2 two times in about 7 days. For example, the first dose of the compound of formula (I) or (Ia) or a compound in Tables 1 or 2 is administered on day 1, and the second dose of the compound of formula (I) or (Ia) or in Tables 1 or 2 may be administered on day 2, day 3, day 4, day 5, day 6 or day 7. In some embodiments, the twice weekly administration occurs on days 1 and 3 or days 1 and 4.

In some embodiments, the compound of formula (I) or (Ia) or in Tables 1 or 2 is cyclically administered twice weekly. For example, the compound of formula (I) or (Ia) or in Tables 1 or 2 is administered for a first period of time, followed by a “dose-free” period, then administered for a second period of time. The language “dose free” includes the period of time in between the first dosing period and the second dosing period in which no compound of formula (I) or (Ia) or in Tables 1 or 2 is administered to the subject. A preferred cycle is administering the compound of formula (I) or (Ia) or in Tables 1 or 2 at a dose described above two times during the week for three consecutive weeks followed by one dose-free week. This cycle is then repeated, as described below.

The language “one cycle” includes the first period of time during which the compound of formula (I) or (Ia) or in Tables 1 or 2 is administered, followed by a dose-free period of time. The dosing cycle can be repeated and one of skill in the art will be able to determine the appropriate length of time for such a cyclical dosing regimen. In one embodiment, the cycle is repeated at least once. In one embodiment, the cycle is repeated two or more times. In one embodiment, the cycle is repeated 3, 4, 5, 6, 7, 8, 9, 10, 11, 12, 13, 14, 15 or more times, or as many times as medically necessary as determined by one of skill in the art. In one embodiment, the cycle is repeated until the patient has been determined to be in partial remission (e.g., 50% or greater reduction in the measurable parameters of tumor growth or complete remission (e.g., absence of cancer). One of skill in the art would be able to determine a patient's remission status using routine methods well known in the art.

It is also found that certain triazolone Hsp90 inhibitors and taxane combinations are surprisingly effective at treating subjects with prostate cancer. The particular combination therapies disclosed herein demonstrate surprising biological activity by showing significant anticancer effects with minimal side effects. In one embodiment, the combination includes the triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, and a taxane. In one embodiment, the combination includes the triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, and docetaxel. In one embodiment, the combination includes the triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, and a taxane. In one embodiment, the combination includes the triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, and docetaxel.

In another embodiment, the combination method of treating a subject with prostate cancer includes the step of administering to the subject an effective amount of an Hsp90 inhibitor described herein and a taxane. In one embodiment, the administration of the Hsp90 inhibitor and the taxane are done concurrently. In another embodiment, the administration of the Hsp90 inhibitor and the taxane are done sequentially. In any one of these embodiments, the taxane may be docetaxel, paclitaxel or Abraxane®. In any one of these embodiments, the Hsp90 inhibitor is a compound represented by formula (I) or (Ia) or a compound in Table 1 or Table 2. In any one of these embodiments, the Hsp90 inhibitor may be the triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof. In any one of these embodiments, the Hsp90 inhibitor may be the triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof.

In one embodiment, the method also includes treating a subject with prostate cancer, comprising administering to the subject an effective amount of a triazolone compound represented by formulae (I) or (Ia), or a compound in Table 1 or Table 2, in combination with an effective amount of paclitaxel or a paclitaxel analogue. In another embodiment, the method includes treating a subject with prostate cancer, comprising administering to the subject an effective amount of paclitaxel or a paclitaxel analogue and an effective amount of the triazolone compound of 3-(2,4-dihydroxy-5-ethyl-phenyl)-4-(1,3-dimethyl-indol-5-yl)-5-hydroxy-[1,2,4]triazole, or a tautomer, or a pharmaceutically acceptable salt thereof.

In another embodiment, the method includes treating a subject with prostate cancer, comprising administering to the subject an effective amount of docetaxel and an effective amount of the triazolone compound of 3-(2,4-dihydroxy-5-ethyl-phenyl)-4-(1,3-dimethyl-indol-5-yl)-5-hydroxy-[1,2,4]triazole, or a tautomer, or a pharmaceutically acceptable salt thereof. In another embodiment, the method includes treating a subject with prostate cancer, comprising administering to the subject an effective amount of paclitaxel or a paclitaxel analogue and an effective amount of the triazolone compound of 3-(2,4-dihydroxy-5-isopropyl-phenyl)-4-(1,3-dimethyl-indol-5-yl)-5-hydroxy-[1,2,4]triazole, or a tautomer, or a pharmaceutically acceptable salt thereof.

In another embodiment, the method includes treating a subject with prostate cancer, comprising administering to the subject an effective amount of docetaxel and an effective amount of the triazolone compound of 3-(2,4-dihydroxy-5-isopropyl-phenyl)-4-(1,3-dimethyl-indol-5-yl)-5-hydroxy-[1,2,4]triazole, or a tautomer, or a pharmaceutically acceptable salt thereof. In yet another embodiment, the method includes treating a subject with prostate cancer, comprising administering to the subject an effective amount of paclitaxel or a paclitaxel analogue and an effective amount of the triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof.

In yet another embodiment, the method includes treating a subject with prostate cancer, comprising administering to the subject an effective amount of paclitaxel or a paclitaxel analogue and a synergistic amount of the triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof. In a further embodiment, the method includes treating a subject with prostate cancer, comprising administering to the subject an effective amount of docetaxel and an effective amount of the triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof.

In a further embodiment, the method includes treating a subject with prostate cancer, comprising administering to the subject an effective amount of docetaxel and a synergistic amount of the triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof. In a further embodiment, the method includes treating a subject with prostate cancer, comprising administering to the subject a synergistic amount of docetaxel and a synergistic amount of the triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof.

In another embodiment, the method includes treating a subject with prostate cancer, comprising administering to the subject an effective amount of paclitaxel or a paclitaxel analogue and an effective amount of the triazolone compound of 3-(2,4-dihydroxy-5-isopropyl-phenyl)-4-(1-isopropyl-indol-4-yl)-5-hydroxy-[1,2,4]triazole, or a tautomer, or a pharmaceutically acceptable salt thereof. In another embodiment, the method includes treating a subject with prostate cancer, comprising administering to the subject an effective amount of docetaxel and an effective amount of the triazolone compound of 3-(2,4-dihydroxy-5-isopropyl-phenyl)-4-(1-isopropyl-indol-4-yl)-5-hydroxy-[1,2,4]triazole, or a tautomer, or a pharmaceutically acceptable salt thereof.

In yet another embodiment, the method includes treating a subject with prostate cancer, comprising administering to the subject an effective amount of paclitaxel or a paclitaxel analogue and an effective amount of the triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof. In yet another embodiment, the method includes treating a subject with prostate cancer, comprising administering to the subject an effective amount of docetaxel and an effective amount of the triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof.

In yet another embodiment, the method includes treating a subject with prostate cancer, comprising administering to the subject an effective amount of docetaxel and a synergistic amount of the triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof. In one embodiment, the compound of formula (I) or (Ia) or in Tables 1 or 2 is administered by intravenous infusion, such as peripheral intravenous infusion. In one embodiment, the compound of formula (I) or (Ia) or in Tables 1 or 2 is infused over 60 minutes.

In some embodiments, the triazolone compound and the paclitaxel analogue are administered concurrently, or separately, or sequentially. In some embodiments, the methods further comprising administering one or more additional therapeutic agents.

In one embodiment, the method includes the use of an Hsp90 inhibitor described herein for the manufacture of a medicament for treating prostate cancer in combination with a taxane. In any one of these embodiments, the taxane may be docetaxel, paclitaxel or Abraxane®. In certain embodiments, the methods described herein provide compositions of Hsp90 inhibitory compounds described herein with a taxane for the treatment of prostate cancer in a subject in need thereof. In any one of these embodiments, the taxane may be docetaxel, paclitaxel or Abraxane®.

In one embodiment, the method includes inhibiting the growth of a cancer or tumor cell, comprising exposing the cell with an effective amount of paclitaxel analogue and an effective amount of a triazolone compound of formulae (I) or (Ia) as defined in claim 1, or a compound in Table 1 or Table 2, or a tautomer or a pharmaceutically acceptable salt thereof.

In one embodiment, the method includes inhibiting the growth of a cancer or a tumor cell in a subject in need thereof, comprising exposing the cell in said subject with an effective amount of paclitaxel analogue and an effective amount of a triazolone compound of formulae (I) or (Ia) as defined in claim 1, or a compound in Table 1 or Table 2, or a tautomer or a pharmaceutically acceptable salt thereof.

In one embodiment, the method includes inhibiting the growth of a cancer or tumor cell, comprising exposing the cell with an effective amount of paclitaxel analogue and an effective amount of a triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof. In one embodiment, the method includes inhibiting the growth of a cancer or tumor cell, comprising exposing the cell with an effective amount of docetaxel and an effective amount of a triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof.

In one embodiment, the method includes inhibiting the growth of a cancer or tumor cell in a subject in need thereof, comprising exposing the cell in said subject with an effective amount of paclitaxel analogue and an effective amount of a triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof. In one embodiment, the method includes inhibiting the growth of a cancer or tumor cell in a subject in need thereof, comprising exposing the cell in said subject with an effective amount of docetaxel and an effective amount of a triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof.

The therapeutic agents of the combination therapies can be administered to a subject, preferably a human subject, in the same pharmaceutical composition. In alternative embodiments, the therapeutic agents of the combination therapies can be administered concurrently to a subject in separate pharmaceutical compositions. The therapeutic agents may be administered to a subject by the same or different routes of administration.

The methods described herein include managing, treating or ameliorating prostate cancer or one or more symptoms thereof in a subject refractory, either completely or partially, to existing agent therapies for prostate cancer, said methods comprising administering to said subject a dose of an effective amount of one or more compounds described herein and a dose of an effective amount of a taxane.

The methods described herein also include treating, managing, or ameliorating prostate cancer or a symptom thereof by administering one or more compounds described herein in combination with a taxane to patients who have proven refractory to other therapies but are no longer on these therapies.

The method described herein is useful for the treatment, and amelioration of prostate cancer. In a specific embodiment, a composition comprising one or more triazolone compounds described herein, or a pharmaceutically acceptable salt thereof, is administered in combination with a taxane. In another embodiment, a composition comprising one or more triazolone compounds described herein, or a pharmaceutically acceptable salt thereof, and one or more other therapeutic agents is administered, in combination with a taxane. Pharmaceutical compositions used herein are formulated to be compatible with its intended route of administration. Examples of routes of administration include parenteral, e.g., intravenous, intradermal, subcutaneous, oral (e.g., inhalation), intranasal, transdermal (topical), transmucosal, and rectal administration. In a specific embodiment, the composition is formulated in accordance with routine procedures as a pharmaceutical composition adapted for intravenous, subcutaneous, intramuscular, oral, intranasal or topical administration to human beings. In a preferred embodiment, a pharmaceutical composition is formulated in accordance with routine procedures for subcutaneous administration to human beings.

Typical pharmaceutical compositions and dosage forms comprise one or more excipients. Suitable excipients are well known to those skilled in the art of pharmacy.

The triazolone compounds described herein can be also formulated into or administered by controlled release means or by delivery devices that are well known to those of ordinary skill in the art. Examples include those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566.

In general, the recommended daily dose range of a triazolone compound for the conditions described herein lie within the range of from about 0.01 mg to about 1000 mg per day, given as a single once-a-day dose preferably as divided doses throughout a day. In one embodiment, the daily dose is administered twice daily in equally divided doses. Specifically, a daily dose range should be from about 5 mg to about 500 mg per day, more particularly, between about 10 mg and about 200 mg per day. In managing the patient, the therapy should be initiated at a lower dose, perhaps about 1 mg to about 25 mg, and increased if necessary up to about 200 mg to about 1000 mg per day as either a single dose or divided doses, depending on the patient's global response. It may be necessary to use dosages of the active ingredient outside the ranges disclosed herein in some cases, as will be apparent to those of ordinary skill in the art. Furthermore, it is noted that the clinician or treating physician will know how and when to interrupt, adjust, or terminate therapy in conjunction with individual patient response.

Different therapeutically effective amounts may be applicable for different subjects, as will be readily known by those of ordinary skill in the art. Similarly, amounts sufficient to prevent, manage, treat or ameliorate such cancers, but insufficient to cause, or sufficient to reduce, adverse effects associated with the triazolone compounds described herein are also encompassed by the above described dosage amounts and dose frequency schedules. Further, when a patient is administered multiple dosages of a triazolone compound described herein, not all of the dosages need be the same. For example, the dosage administered to the patient may be increased to improve the prophylactic or therapeutic effect of the compound or it may be decreased to reduce one or more side effects that a particular patient is experiencing.

In a specific embodiment, the dosage of the composition comprising a triazolone compound described herein administered to prevent, treat, manage, or ameliorate cancer, or one or more symptoms thereof in a patient is 150 μg/kg, preferably 250 μg/kg, 500 μg/kg, 1 mg/kg, 5 mg/kg, 10 mg/kg, 25 mg/kg, 50 mg/kg, 75 mg/kg, 100 mg/kg, 125 mg/kg, 150 mg/kg, or 200 mg/kg or more of a patient's body weight. In another embodiment, the dosage of the composition comprising a compound described herein administered to prevent, treat, manage, or ameliorate cancer, or one or more symptoms thereof in a patient is a unit dose of 0.1 mg to 20 mg, 0.1 mg to 15 mg, 0.1 mg to 12 mg, 0.1 mg to 10 mg, 0.1 mg to 8 mg, 0.1 mg to 7 mg, 0.1 mg to 5 mg, 0.1 to 2.5 mg, 0.25 mg to 20 mg, 0.25 to 15 mg, 0.25 to 12 mg, 0.25 to 10 mg, 0.25 to 8 mg, 0.25 mg to 7 m g, 0.25 mg to 5 mg, 0.5 mg to 2.5 mg, 1 mg to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg. The unit dose can be administered 1, 2, 3, 4 or more times daily, or once every 2, 3, 4, 5, 6 of 7 days, or once weekly, once every two weeks, once every three weeks or once monthly.

In certain embodiments, when the triazolone compounds described herein are administered in combination with a taxane, the therapies are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In one embodiment, two or more therapies are administered within the same patient visit.

In certain embodiments, one or more compounds described herein and a taxane are cyclically administered. Cycling therapy involves the administration of a first therapy (e.g., a first prophylactic or therapeutic agents) for a period of time, followed by the administration of a second therapy (e.g., a second prophylactic or therapeutic agents) for a period of time, followed by the administration of a third therapy (e.g., a third prophylactic or therapeutic agents) for a period of time and so forth, and repeating this sequential administration, i.e., the cycle in order to reduce the development of resistance to one of the agents, to avoid or reduce the side effects of one of the agents, and/or to improve the efficacy of the treatment.

In certain embodiments, administration of the same compound described herein may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same prophylactic or therapeutic agent may be repeated and the administration may be separated by at least at least 1 day, 2 days, 3 days, 5 days, 10 days, 15 days, 30 days, 45 days, 2 months, 75 days, 3 months, or 6 months.

In a specific embodiment, a method of preventing, treating, managing, or ameliorating a proliferative disorders, such as prostate cancer, or one or more symptoms thereof, said methods comprising administering to a subject in need thereof a dose of at least 150 μg/kg, preferably at least 250 μg/kg, at least 500 μg/kg, at least 1 mg/kg, at least 5 mg/kg, at least 10 mg/kg, at least 25 mg/kg, at least 50 mg/kg, at least 75 mg/kg, at least 100 mg/kg, at least 125 mg/kg, at least 150 mg/kg, or at least 200 mg/kg or more of one or more compounds described herein once every day, preferably, once every 2 days, once every 3 days, once every 4 days, once every 5 days, once every 6 days, once every 7 days, once every 8 days, once every 10 days, once every two weeks, once every three weeks, or once a month. Alternatively, the dose can be divided into portions (typically equal portions) administered two, three, four or more times a day.

In one embodiment, the taxane (e.g., paclitaxel or docetaxel) is administered once every 3 weeks schedule and the Hsp inhibitor (e.g., ganetespib) is administered on week 1 and 2 (with a rest week after that) before beginning again. Alternatively, a once every three week regimen at a starting dose of 60 mg/m2 escalating to 75 mg/m2 for paclitaxel or docetaxel is used. In another alternative, a 3 week on/1 week off regimen starting at 30 mg/m2 escalating to 35 mg/m2 with paclitaxel or docetaxel is used. The amount of the Hsp 90 inhibitor is adjusted according to tolerability and efficacy, as described above.

In another alternative, paclitaxel is given either once weekly (typical dose 90 mg/m², range 70-100). Alternatively it is given once every three weeks. Doses range from 175 to 225 mg/m² when given once every three weeks. The dose of the Hsp 90 inhibitor is commonly a full single agent dose (e.g., 200 mg/m², or less, depending on tolerability, as described above.

In another alternative, docetaxel is given once every three weeks (dose level 75 mg/m², range 60-100 mg/m²). It can be also given weekly, range 30-40 mg/m². The dose of the Hsp 90 inhibitor is commonly a full single agent dose (e.g., 200 mg/m², or less, depending on tolerability, as described above.

Alternatively, the treatment cycle comprises weekly treatments for 2 weeks followed by a 1-week rest period. Treatment cycles will be repeated every 3 weeks. The Hsp90 inhibitor is administered (150 mg/m² or 200 mg/m²) on Days 1 and 8 of each cycle and docetaxel (60 mg/m² or 75 mg/m²) is administered on Day 1 of each cycle. The treatment is repeated every three weeks.

In another alternative, subjects are administered 200 mg/m² of the Hsp90 inhibitor followed by docetaxel 25 mg/m², 30 mg/m or 35 mg/m² for three consecutive weeks followed by a 1-week dose-free interval. Treatment is then repeated.

The dosages of prophylactic or therapeutic agents other than compounds described herein, which have been or are currently being used to prevent, treat, manage, or proliferative disorders, such as cancer, or one or more symptoms thereof can be further used in the combination therapies described herein. Preferably, dosages lower than those which have been or are currently being used to prevent, treat, manage, or ameliorate a proliferative disorder, or one or more symptoms thereof, are used in the combination therapies described herein. The recommended dosages of agents currently used for the prevention, treatment, management, or amelioration of a proliferative disorders, such as cancer, or one or more symptoms thereof, can obtained from any reference in the art including Hardman et al., eds., 1996, Goodman & Gilman's The Pharmacological Basis Of Basis Of Therapeutics 9^{th} Ed, Mc-Graw-Hill, New York; Physician's Desk Reference (PDR) 57^{th} Ed., 2003, Medical Economics Co., Inc., Montvale, N.J.

In another embodiment, the method also includes monitoring the treatment response of a subject with prostate cancer being treated with a compound of formula (I) or (Ia) or a compound in Tables 1 or 2, comprising (a) determining the level of maspin in a biological sample derived from the subject before the treatment of said compound; (b) determining the level of maspin in a biological sample derived from the subject at a time point during or after administration of the compound; and (c) comparing the level of maspin in the biological sample derived from the subject during or after treatment with that before the treatment, wherein an increase of the maspin level in the biological sample is indicative of a positive response to the treatment of the triazolone compound. In one embodiment, the subject may be treated with the triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof. In one embodiment, the subject may be treated with the triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof.

In another embodiment, the method also includes monitoring the treatment response of a subject with prostate cancer being treated with the triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, comprising (a) determining the level of maspin in a biological sample derived from the subject before the treatment of said compound; (b) determining the level of maspin in a biological sample derived from the subject at a time point during or after administration of the compound; and (c) comparing the level of maspin in the biological sample derived from the subject during or after treatment with that before the treatment, wherein an increase of the maspin level in the biological sample is indicative of a positive response to the treatment of the triazolone compound.

In another embodiment, the method also includes monitoring the treatment response of a subject with prostate cancer being treated with the triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, comprising (a) determining the level of maspin in a biological sample derived from the subject before the treatment of said compound; (b) determining the level of maspin in a biological sample derived from the subject at a time point during or after administration of the compound; and (c) comparing the level of maspin in the biological sample derived from the subject during or after treatment with that before the treatment, wherein an increase of the maspin level in the biological sample is indicative of a positive response to the treatment with the triazolone compound.

In any one of the above embodiments, the prostate cancer may be metastatic prostate cancer, metastatic hormone-resistant prostate cancer or metastatic castration-resistant prostate cancer, or prostate cancer wherein it was previously treated with docetaxel-based chemotherapy. In any one of these embodiments, the biological sample is a serum sample derived from the subject.

The following examples are intended only as illustrative for the embodiments described herein, and should not be interpreted to be limiting the scope of the invention in any way.

### EXAMPLES

### Example 1

Materials and Methods

The LNCaP, VCaP, 22Rv1, DU145 and PC3 human prostate cancer cell lines and HeLa cells were all purchased from the American Type Culture Collection (Manassas, Va., USA). Cells were maintained and cultured according to standard techniques at 37° C. in 5% (v/v) CO₂ using culture medium recommended by the supplier. All primary antibodies were purchased from Cell Signaling Technology (Beverly, Mass., USA) with the exception of RAF1 (Santa Cruz Biotechnology, Santa Cruz, Calif., USA), p-EGFR (Tyr1068) (Invitrogen, Carlsbad, Calif., USA) and actin (GE Healthcare, UK). The Hsp90 inhibitors ganetespib and 17-AAG were synthesized at Synta Pharmaceuticals Corp. methyltrienolone (R1881) was purchased from Perkin-Elmer (Boston, Mass., USA).

Cell Viability Assays

Cellular viability was assessed using the CellTiter-Glo Luminescent Cell Viability Assay (Promega, Madison, Wis., USA) according to the manufacturer's protocol. Twenty-four hours after plating at 5×10³ cells/well in triplicate in 96-well plates, cells were dosed with graded concentrations of ganetespib or 17-AAG for 72 h. CellTiter-Glo was added (50% v/v) to the cells, and the plates incubated for 10 min prior to luminescent detection in a SpectraMax Plus 384 microplate reader (Molecular Devices, Sunnyvale, Calif., USA). Data were normalized to percent of control and IC₅₀ values used to determine the sensitivity of each line.

Western Blotting

Prostate cancer cell lines were lysed in RIPA buffer (Cell Signaling Technology, Beverly, Mass. USA) and HeLa lysed by four rounds of freeze/thawing using 1× Reporter Lysis Buffer (Promega, Madison, Wis., USA) containing 0.4 M NaCl. Lysates were clarified by centrifugation and equal amounts of protein resolved by SDSPAGE before transfer to nitrocellulose membranes. Membranes were blocked with 5% skim milk in TBS with 0.5% Tween and immunoblotted with indicated antibodies. Antigen-antibody complexes were visualized using an Odyssey system (LI-COR, Lincoln, Nebr., USA).

Quantitative RT-PCR

LNCaP cells were cultured in charcoal-stripped medium for 24 h and then treated with 250 nM ganetespib, 1 μM geldanamycin, or vehicle for 24 h in the absence or presence of 10 nM methyltrienolone (R1881). RNA was prepared from the LNCaP cells post-treatment using TRIzol reagent (Invitrogen, Grand Island, N.Y., USA). Previously reported prostate specific antigen (PSA), transmembrane protease, serine 2 (TMPRSS2), and 18S primer sets were used for target gene expression and were analyzed using SYBR green PCR Master mix in an ABI 7500 Fast sequence detection system. See, e.g., Agoulnik et al (2006), Androgens modulate expression of transcription intermediary factor 2, an androgen receptor coactivator whose expression level correlates with early biochemical recurrence in prostate cancer. Cancer Res 66 10594-10602. PSA and TMPRSS2 mRNA levels were normalized to 18S mRNA values.

Transient Transfection of HeLa Cells

HeLa cells were transiently transfected using a poly-L-lysine coupled adenoviral-mediated DNA transfer technique as previously described. See, e.g., Nazareth et al (1996), Activation of the human androgen receptor through a protein kinase A signaling pathway. J Biol Chem 271 19900-19907. The plasmid constructs used were pCR3.1-AR (encoding for full length AR) and pCR3.1-V7 (encoding for the V7 truncated AR isoform and which was a gift from Manjula Nakka and William Krause, Baylor College of Medicine). For the expression study, HeLa cells were transfected with 3 ng of pCR3.1-AR or 0.5 ng of pCR3.1-V7 for 24 h. Cells were treated with R1881 (10 nM), GA (1 μM), and/or STA9090 (250 nM) or vehicle (ethanol and DMSO) for 24 hours prior to lysis and immunoblotting. To determine the effect of Hsp90 inhibitors on AR and variant activity, HeLa cells were transiently transfected with 250 ng of GRE-luciferase reporter, 30 ng of pCR3.1 β-galactosidase, 3 ng of pCR3.1-AR, or 0.03 ng of pCR3.1-V7 and treated as above. Luciferase and β-galactosidase activities were measured and luciferase levels normalized to β-galactosidase levels as described previously. See, e.g., Agoulnik et al (2003), Repressors of androgen and progesterone receptor action. J Biol Chem 278 31136-31148.

Flow Cytometry

For cell cycle analysis, PC3 and DU145 cells were seeded overnight at 0.3×10⁶ cells/5 mL in a 6-well plate and then exposed to increasing concentrations of ganetespib (0-500 nM) for 24 h. Cells were harvested and stained with propidium iodide using the BD Cycle TEST PLUS Reagent Kit (BD Biosciences, San Jose, Calif., USA) according to the manufacturer's instructions. Twenty thousand cells were analyzed for their DNA content using a FACS Caliber cytometer (BD Biosciences, Billerica, Mass., USA). For the apoptosis assay in the DU145 cell line, cells were treated with ganetespib (10, 100 or 500 nM), 17-AAG (500 or 1000 nM) or control (DMSO) for 24 h. Following treatment cells were harvested and stained using a fluorescein-conjugated anti-Annexin V antibody (BD Biosciences) and apoptosis assessed by flow cytometry.

In Vivo Prostate Xenograft Model

Eight-week-old female immunodeficient nude mice (Charles River Laboratories, Wilmington, Mass.) were maintained in a pathogen-free environment, and all in vivo procedures were approved by the Synta Pharmaceuticals Corp. Institutional Animal Care and Use Committee. PC3 tumor cells were subcutaneously implanted into nude mice. Mice bearing established tumors (100-200 mm³) were randomized into treatment groups of 8 and i.v. dosed via the tail vein with either vehicle or ganetespib formulated in 10/18 DRD (10% DMSO, 18% Cremophor RH 40, 3.6% dextrose, 68.4% water). Tumor volumes (V) were calculated by caliper measurements of the width (W), length (L), and thickness (T) of each tumor using the formula: V=0.5236(LWT). Tumor growth inhibition was determined as described previously. See Proia et al (2011), Multifaceted intervention by the Hsp90 inhibitor ganetespib (STA-9090) in cancer cells with activated JAK/STAT signaling. PLoS One 6 e18552.

Experimental Results

Ganetespib Potently Induces Cell Death in Prostate Cancer Cells Irrespective of Androgen Receptor Status

Initial examination was performed on the growth inhibitory effects of ganetespib in vitro using a panel of prostate cancer cell lines. In all cases, ganetespib reduced cell viability in a dose-dependent manner and was more potent than the first-generation ansamycin Hsp90 inhibitor 17-AAG (Table 1).

| | AR expression/ | Ganetespib | 17-AAG |
|---|---|---|---|
| Cell line | Androgen sensitivity | (nM) | (nM) |
| LNCaP | +/Dependent | 8 | 265 |
| VCaP | +/Dependent | 7 | 2645 |
| 22Rv1 | +/Partial | 2 | 1270 |
| DU145 | −/Independent | 12 | 36 |
| PC3 | −/Independent | 77 | 246 |

In the AR-negative cell lines DU145 and PC3 the cytotoxicty IC₅₀ values at 72 h were 12 and 77 nM, respectively. The AR-positive, androgen-dependent cell lines LNCaP and VCaP were more sensitive to ganetespib exposure (IC₅₀ values of 8 and 7 nM). The 22Rv1 cell line, which while AR-positive is only weakly androgen responsive, manifested the greatest sensitivity to ganetespib (IC₅₀, 2 nM). These data demonstrate that Hsp90 inhibition by ganetespib resulted in potent cytotoxic effects in prostate cancer lines regardless of their AR status or androgen sensitivity.

Coordinate Inhibition of AR Activity and Multiple Oncogenic Signaling Pathways in Prostate Cancer Cells by Ganetespib

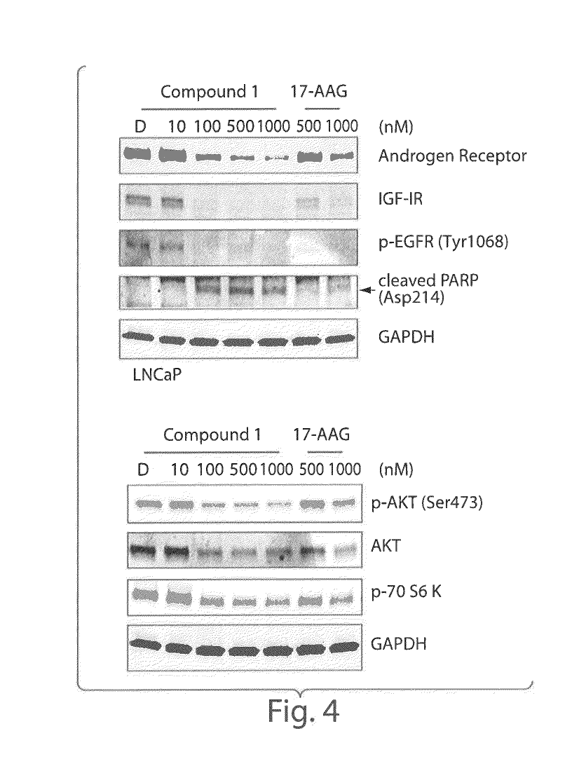

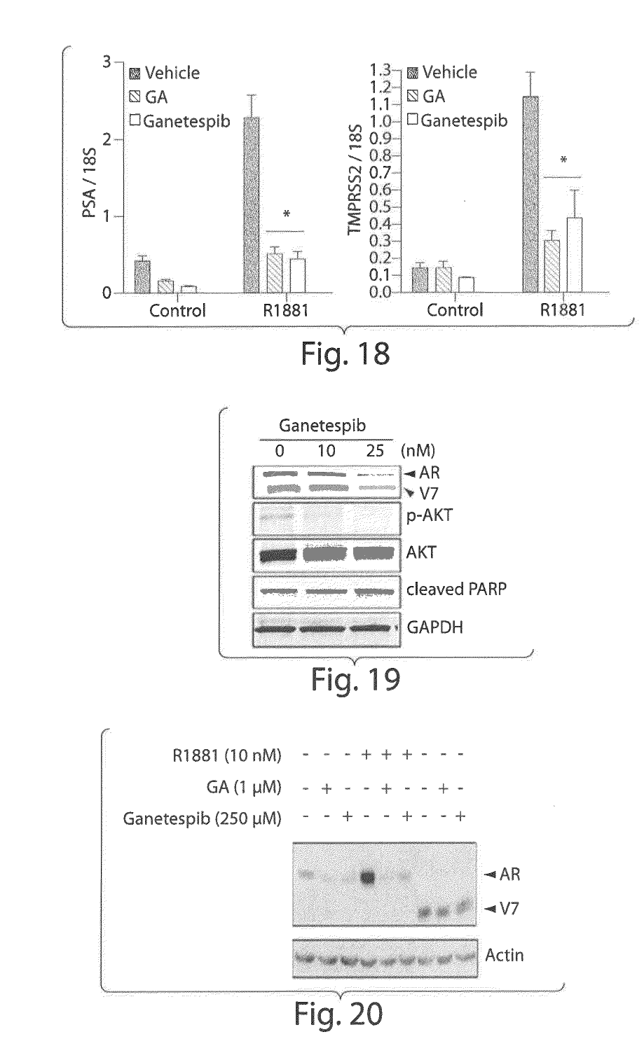

Targeted degradation of client proteins is a feature of Hsp90 inhibition. Expression changes were examined in Hsp90 clients known to be associated with prostate tumor progression. AR-positive LNCaP cells were treated with ganetespib or 17-AAG for 24 h and protein levels determined by Western blot (FIG. 4). Ganetespib treatment resulted in a potent and dose-dependent decrease in AR levels. In control-treated LNCaP cells, AR staining was primarily compartmentalized to the nucleus. After a 4 h exposure to ganetespib (100 nM) the intensity of staining was significantly diminished, nuclear localization lost and only faint cytoplasmic staining was detectable. Hsp90-directed loss of AR receptor expression resulted in consequent suppression of AR-directed gene regulation. To show this, LNCaP cells were cultured in charcoal-stripped medium for 24 h and then treated with ganetespib, geldanamycin (GA, the parent compound from which 17-AAG is derived), or vehicle for 24 h in the absence or presence of androgen (R1881). As a read-out of AR-specific transcriptional activity, prostate specific antigen (PSA) and transmembrane protease, serine 2 (TMPRSS2) mRNA levels were measured and normalized to 18S mRNA values (FIG. 18). In accordance with the androgen-inducible expression of both genes, R1881 exposure increased PSA and TMPRSS2 levels in control cells. This induction was significantly inhibited in the presence of either Hsp90 inhibitor (*p<0.001) (FIG. 18).

Importantly, ganetespib also induced degradation of IGF-IR and phosphorylated EGFR receptors, previously implicated in the pathogenesis of prostate cancer, as well as the downstream effectors AKT and p70 S6K, in LNCaP cells (FIG. 4). Moreover a concomitant increase in PARP cleavage, a marker of apoptosis, accompanied the reductions in these protein levels. Consistent with the differences in sensitivity shown in Table 1, ganetespib was comparatively more potent than 17-AAG at inducing targeted loss of these oncogenic proteins and signaling pathways.

Constitutively Active AR Variant Expression does not Confer Resistance to Ganetespib

The expression of alternatively spliced, terminally-truncated AR isoforms is one potential mechanism for the development of a castration-resistant phenotype. See, e.g., Dehm et al (2008), Splicing of a novel androgen receptor exon generates a constitutively active androgen receptor that mediates prostate cancer therapy resistance. Cancer Res 68 5469-5477. For example, the 22Rv1 cell line expresses the full-length AR protein as well as constitutively active variants that lack the carboxyl-terminal ligand-binding domain, thereby reducing its dependence on exogenous androgen. See, e.g., Hu et al (2009), Ligand-independent androgen receptor variants derived from splicing of cryptic exons signify hormone-refractory prostate cancer. Cancer Res 69 16-22; Li et al (2011), Intragenic rearrangement and altered RNA splicing of the androgen receptor in a cell-based model of prostate cancer progression. Cancer Res 71 2108-2117. Of note, it was found that 22Rv1 cells were acutely sensitive to the effects of ganetespib treatment (Table 1). As shown in FIG. 18, even a 25 nM dose of ganetespib was sufficient to induce degradation of full-length AR, destabilization of p-AKT/AKT activity, and apoptosis (cleaved PARP). In contrast, expression of the truncated isoform (corresponding to the known V7 variant; was less affected by Hsp90 inhibition (FIG. 19). To extend this finding, transient transfection was done from full-length and V7 receptors into HeLa cells (FIG. 20). Androgen treatment increased full-length AR expression at 24 h and this response was completely abrogated in the presence of either 1 mM GA or 250 nM ganetespib. Both Hsp90 inhibitors were also effective at targeted degradation of AR in the absence of androgen stimulation, however neither inhibitor significantly altered expression of the variant receptor (FIG. 20). Similarly, GA and ganetespib strongly inhibited full-length AR activity but were less effective against constitutive V7 activity as shown by luciferase assay. Although the truncated AR isoform appears less sensitive to Hsp90 inhibition, the potent activity of ganetespib in this line suggests that its concomitant impacts on multiple signaling pathways can overcome the selective advantages provided by constitutively active variant expression.

Ganetespib Inhibited Multiple Oncogenic Hsp90 Client Proteins in AR-Negative Prostate Cancer Cells to Induce Cell Death

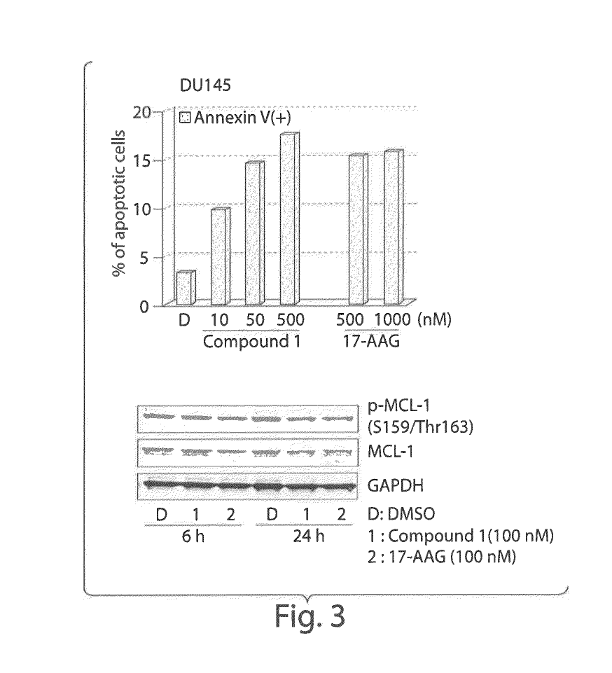

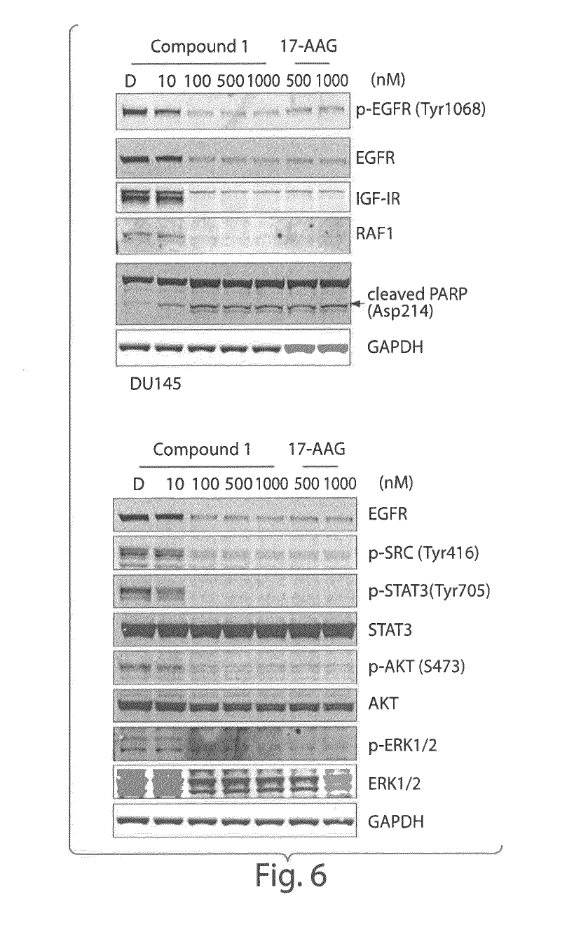

The androgen-independent DU145 cell line lacks AR receptor expression. However the growth and survival of these cells has been reported to be regulated through autocrine activation of EGFR by its ligands, in turn leading to oncogenic STAT activation. See, e.g., Connolly et al (1991), Autocrine regulation of DU145 human prostate cancer cell growth by epidermal growth factor-related polypeptides. Prostate 19 173-180. Further, this line also expresses an autocrine IL-6 cytokine signaling loop that results in persistent activation of the JAK/STAT signaling pathway. Ganetespib effectively targeted EGFR and completely abrogated STAT3 signaling in these cells in a dose-dependent manner (FIG. 6). In addition, IGF-IR and downst1beam signaling pathways mediated through p-AKT, RAF1, and p-ERK1/2 were also destabilized following ganetespib exposure, similar to what was observed in LNCaP cells (FIG. 3). The correlative increase in cleaved PARP expression indicated that simultaneous blockade of these signaling pathways triggered apoptosis and this was further supported by Annexin V staining (FIG. 3). Cells were treated with escalating doses of ganetespib or 17-AAG for 24 h and then analyzed by flow cytometry. Ganetespib treatment resulted in a dose-dependent increase in apoptotic cells. A comparable proportion of apoptotic cells was seen following high doses of 17-AAG, a response that was saturated by the 500 nM exposure level.

Kinetics of Hsp90 Client Protein Degradation by Ganetespib

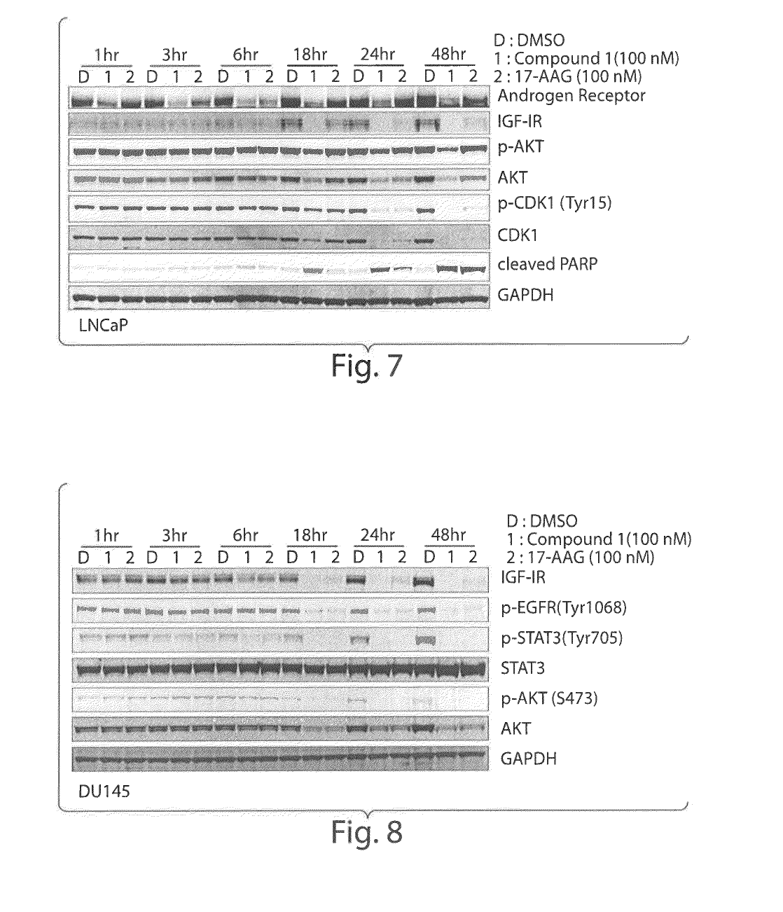

Experiments were also conducted for the kinetics of client protein loss in response to Hsp90 inhibition. In LNCaP cells, 100 nM ganetespib treatment rapidly (within 3 h) resulted in a measurable reduction in AR expression and this effect was sustained over a 48 h time course (FIG. 7). Destabilization of p-AKT/AKT was a relatively later event occurring at 18 h and whose kinetics matched those observed for the elevation of cleaved PARP. Interestingly, ganetespib also induced a temporal loss of both the total and phosphorylated forms of cyclin dependent kinase 1 (CDK1), a key regulator of the G₂/M checkpoint, by 24 h and this effect persisted until at least 48 h (FIG. 7).

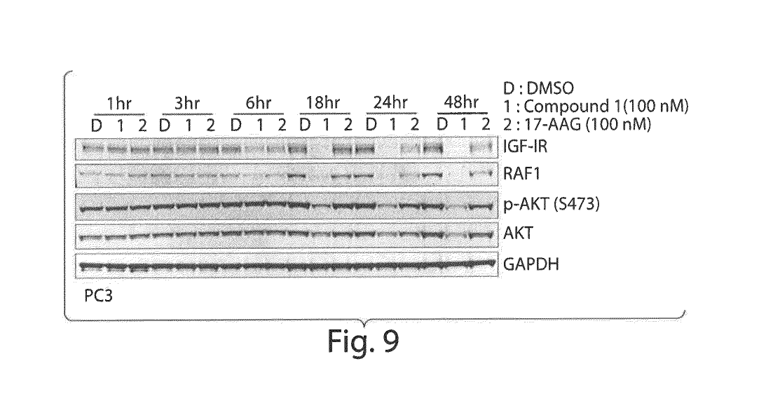

The kinetics of targeted AKT degradation was similar in the AR-negative prostate cell lines DU145 and PC3 (FIGS. 8 and 9, respectively). In DU145 cells, significant reductions in p-EGFR expression also required an 18 h exposure to either ganetespib or 17-AAG, whereas destabilization of IGF-IR and p-STAT3 was evident by 6 h (FIG. 8). Like LNCaP cells, PC3 prostate cells were significantly more sensitive to the effects of ganetespib treatment compared to an equivalent dose of 17-AAG (FIG. 9). Consistent with the DU145 results, ganetespib reduced IGF-1R levels in this line by 6 h and sustained loss of the receptor was observed over the 48 h time course. In addition, a potent and time-dependent reduction in RAF1 protein expression which also preceded AKT modulation was observed (FIG. 9).

Modulation of Cell Cycle Protein Expression by Ganetespib Induces Growth Arrest and Apoptosis

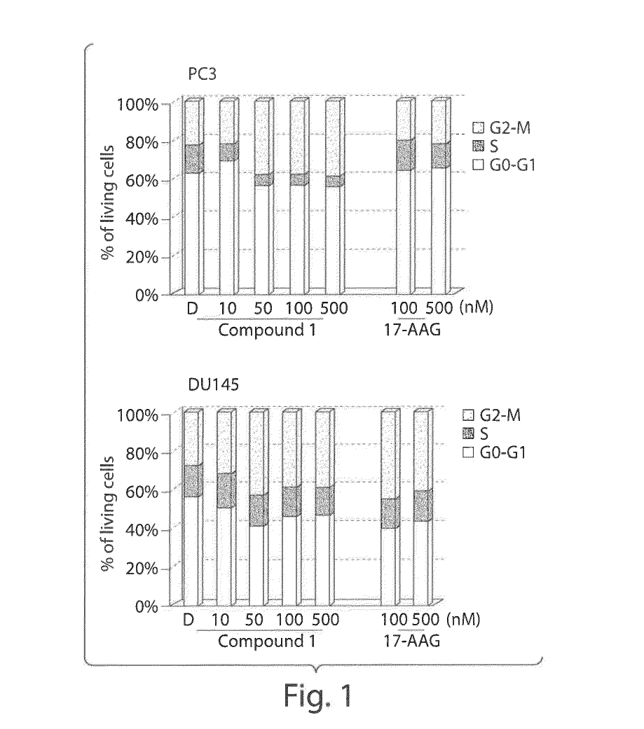

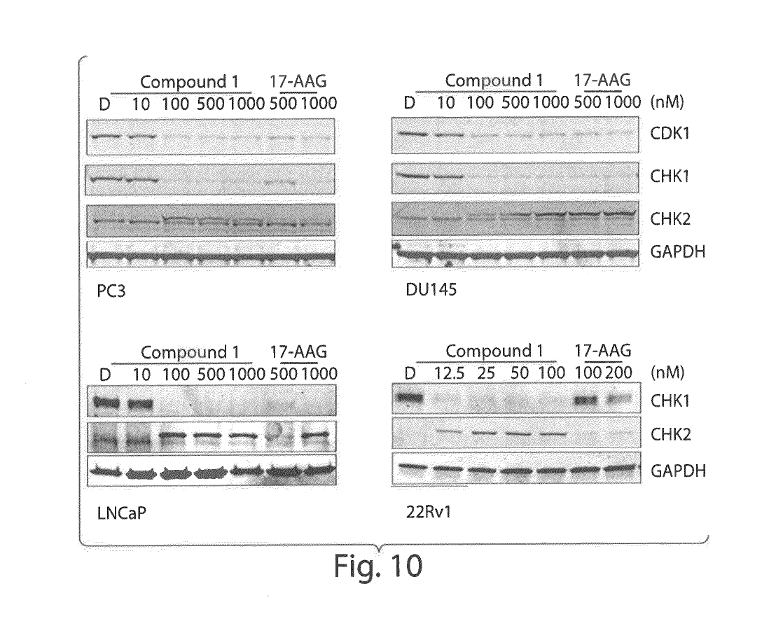

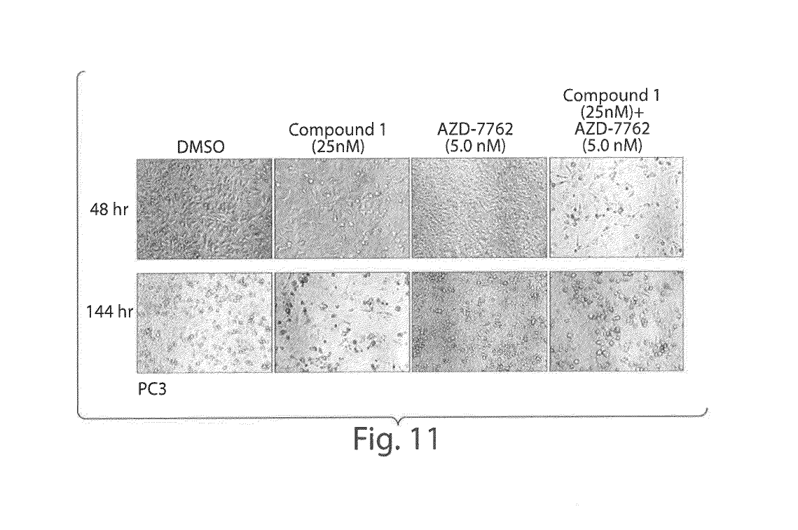

It was previously reported that ganetespib treatment can exert profound effects on cell cycle regulatory proteins, in addition to oncogenic signaling pathways, that contribute to its antitumor activity. See, Proia et al (2011), Multifaceted intervention by the Hsp90 inhibitor ganetespib (STA-9090) in cancer cells with activated JAK/STAT signaling. PLoS One 6 e18552. Cell cycle analysis revealed that ganetespib exposure led to a dose-dependent accumulation of cells in the G₂/M phase in both DU145 and PC3 cells, with a concomitant loss of S phase (FIG. 1). In both cell lines, it was observed a corresponding reduction in protein expression of CDK1 as well as CHK1, another kinase that plays an essential role in the integrity of the G₂/M checkpoint (FIG. 10). Next, more extensive characterization of the concomitant impact of ganetespib on both oncogenic and cell cycle signaling was performed in androgen-dependent VCaP prostate cells. As seen in the LNCaP line (FIG. 4), ganetespib treatment of these cells induced AR and IGF-IR degradation and reduced p-AKT/AKT levels in a dose-dependent manner (FIG. 4). In addition, loss of both the total and phosphorylated forms of CDK1 was observed as a function of dose. Taken together, these data suggest that loss of checkpoint control and G₂/M arrest accompanies blockade of oncogenic signaling in prostate cancer cells as a result of Hsp90 inhibition by ganetespib. Moreover, we observed concomitant elevations in phosphorylated histone H2AX and PARP cleavage (FIG. 4). Since the phosphorylated form of H2AX is a sensitive indicator of DNA double strand break formation, these data suggest that G₂/M arrest leads to subsequent apoptosis.

Ganetespib Inhibits Androgen-Independent PC3 Tumor Growth In Vivo

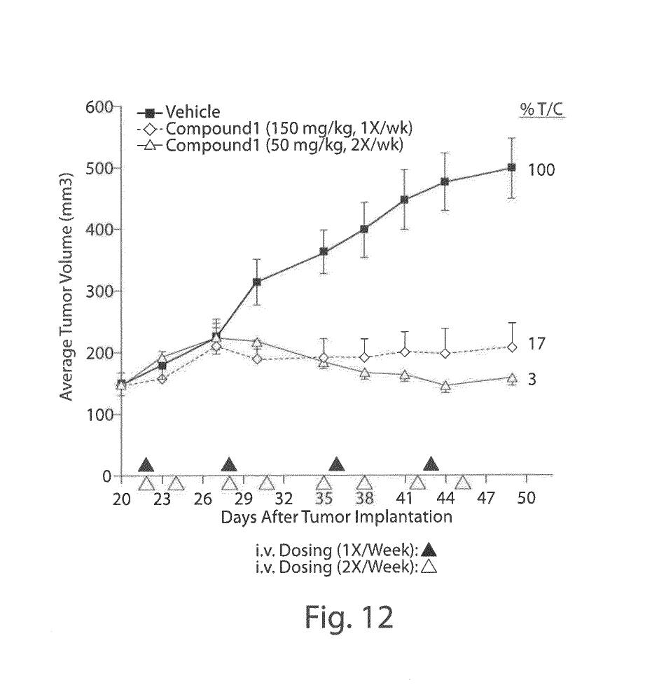

Finally, efficacy studies were conducted of single-agent ganetespib treatment on the growth of PC3 tumor xenografts to determine whether the potent in vitro effects of ganetespib translated to in vivo antitumor activity. It has previously been determined that the highest non-severely toxic dose (HNSTD) of ganetespib on a weekly dosing regimen is 150 mg/kg. See Ying et al (2011), Ganetespib, a unique triazolone-containing Hsp90 inhibitor, exhibits potent antitumor activity and a superior safety profile for cancer therapy. Mol Cancer Ther 2012; 11:475-484. As shown in FIG. 12, mice treated on this schedule exhibited a significant decrease in tumor volume compared to control animals (T/C value 17%). Even at this dose the regimen was well tolerated. These data show that ganetespib treatment can significantly inhibit androgen-independent tumor growth.

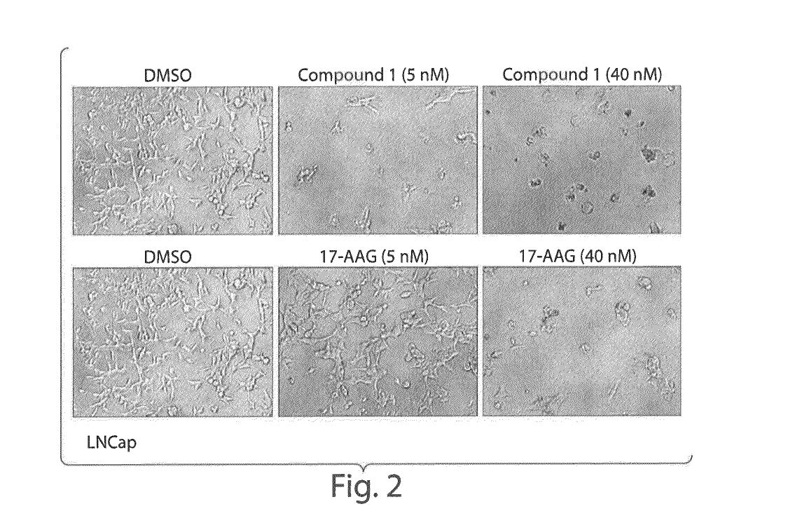

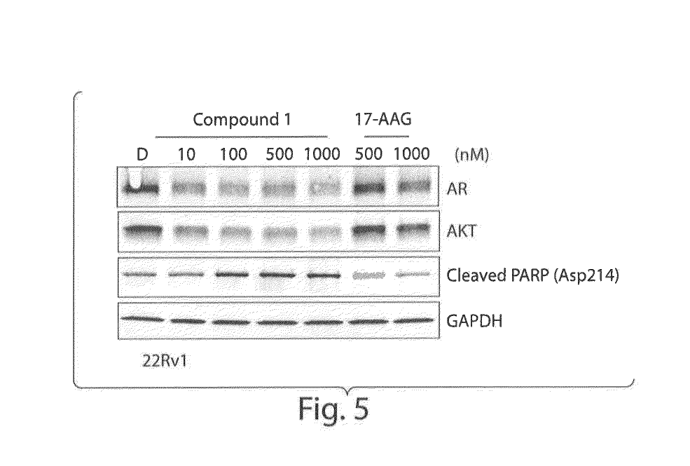

In short summary, the effectiveness of ganetespib or the first generation Hsp90 inhibitor 17-AAG was examined in both hormone-dependent (LNCaP, 22Rv1) and hormone-independent (PC-3, DU-145) PCa cell lines. Ganetespib displayed low nanomolar activity regardless of the cell's AR status, with IC50's 3-7 fold less than 17-AAG. In the treated cultures, ganetespib increased the population of apoptotic (Annexin V positive) cells, whose appearance paralleled the dose-dependent degradation of the anti-apoptotic protein Mcl-1. In all of the cell lines, the master cell cycle regulator Cdk1 and the DNA damage checkpoint protein Chk1 were completely destabilized by ganetespib exposure. This led to the cells arresting in G2/M (FIGS. 1-10). Interestingly, expression of a distinct isoform of Chk2 was enhanced in response to the down-regulation of Chk1, suggesting a potential feedback loop. Also evaluated was the stability of several client proteins (AR, IGF-1R, EGFR, RAF1 and JAK2) and their effectors responsible for PCa progression in response to ganetespib, and significant degradation/inactivation was observed, albeit with variable kinetics. PC3 xenografts were implanted in nude mice, followed by treatment with ganetespib once a week at 150 mg/kg or twice a week at 50 mg/kg for 4 weeks. Ganetespib displayed potent single agent activity versus vehicle, with % T/C values of 17, 3 respectively (FIG. 12). In conclusion, ganetespib is a highly potent Hsp90 inhibitor that displayed preclinical activity in a panel of prostate cancer cell lines due to its ability to target the key signaling components required for PCa cell growth, survival and cell division.

### Example 2

### Combination Treatment of Ganetespib and Docetaxel in Prostate Cancer Cells

Materials and Methods

Cell Lines

Human DU-145 prostate carcinoma cells (American Type Culture Collection) were grown in Dulbecco's modified Eagle's medium with 4 mM L-glutamine, antibiotics (100 IU/ml penicillin and 100 μg/ml streptomycin) and 10% fetal bovine serum (Sigma Aldrich). Cells were maintained at 37° C., 5% CO₂ atmosphere.

Cell Viability Assays

Cell viability was measured using the alamarBlue assay (Invitrogen). In brief, cells were plated in 96-well plates in triplicate at 5000 cells per and incubated at 37° C., 5% CO₂ atmosphere for 24 hr prior to the addition of drug or vehicle (0.3% DMSO) to the culture medium. After 72 hr, 10 μl/well alamarBlue was added to the wells and incubated for an additional 3 hr at 37° C., 5% CO₂ atmosphere. Fluorescence (560_{EX}/590_{EM} nM) was measured with a SpectraMax microplate reader (Molecular Devices) and the resulting data were used to calculate cell viability, normalized to vehicle control.

Combination Studies with Docetaxel and Ganetespib

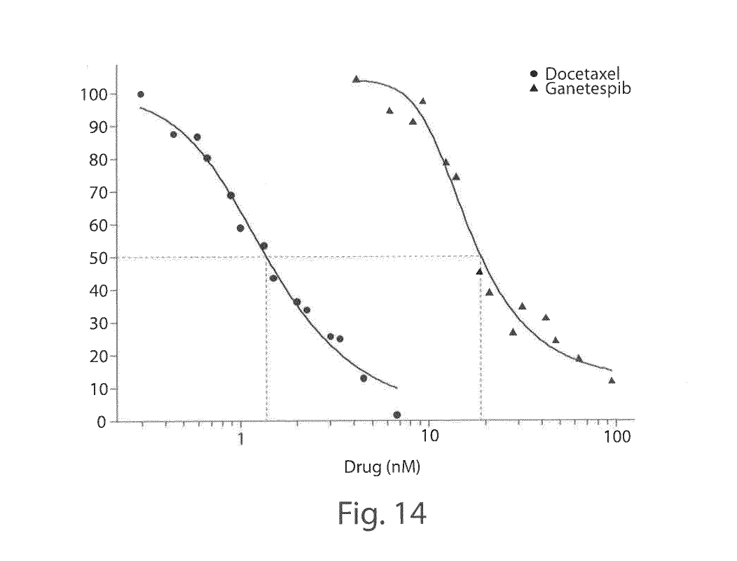

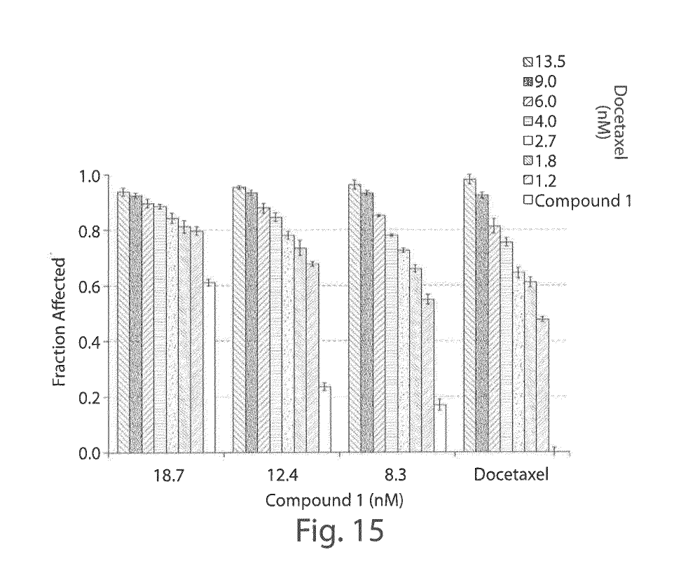

The half maximal inhibitory concentration (IC₅₀) for docetaxel or Ganetespib was determined using a three-fold serial dilution series of compound starting with a top concentration of 1 μM. After 72 hr exposure to drug, cell viability was measured. Data were used to calculate the IC₅₀ values using XLFit software (ID Business Solutions). From the results, Ganetespib has an IC₅₀ of 14 nM, docetaxel has an IC₅₀ of 1 nM in DU-145 cells, shown in FIG. 14. Combinations between docetaxel and Ganetespib were then performed concurrently based on the IC₅₀ for each agent. The combined drugs, as well as each drug alone, were incubated with the cells for 3 days and the surviving fraction of cells relative to control was determined using the alamarBlue assay. Shown in FIG. 15, the combination of docetaxel with Ganetespib displayed enhanced cytotoxicity relative to either agent alone at low doses of compound.

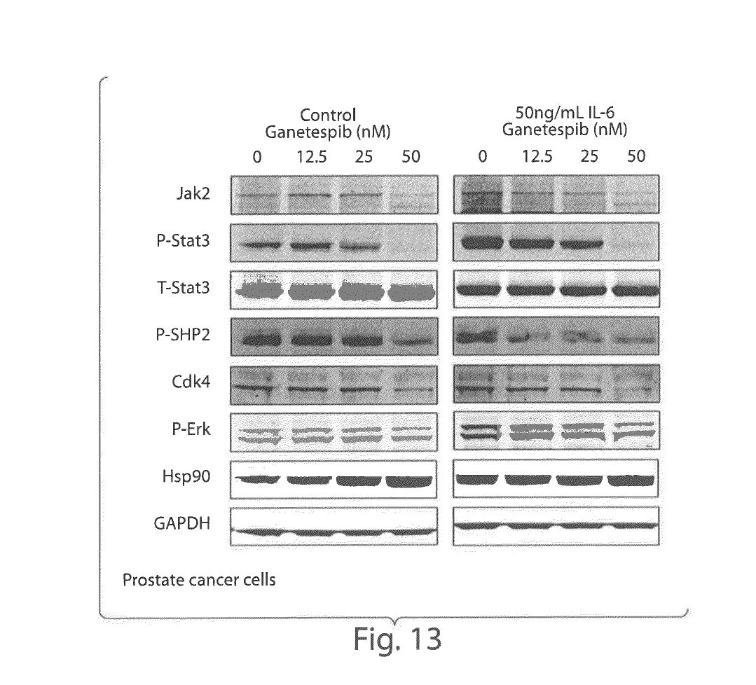

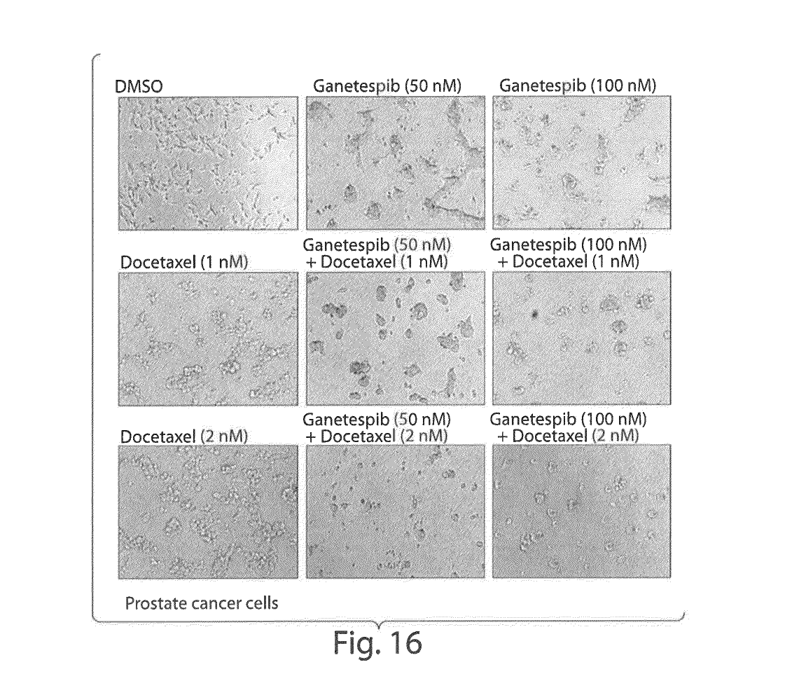

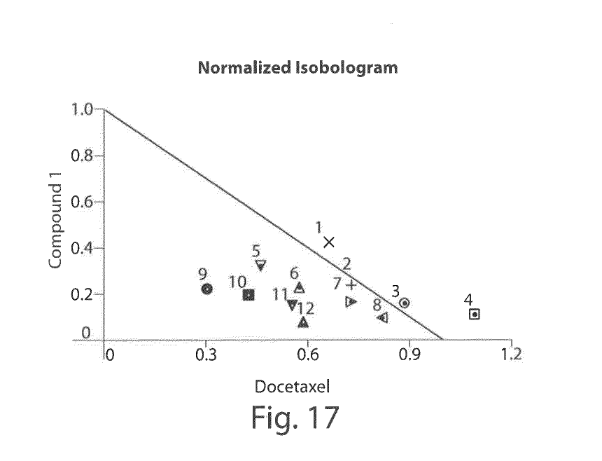

Data from the combination experiment was then used to derive combination index values using the median effect analysis software CalcuSyn 2.0 (CalcuSyn, Inc.). A normalized isobologram was then established and showed that the majority of combination points were synergistic, with the line establishing an additive relationship and those points above the line reflect antagonism, as shown in FIG. 17. These experiments showed clear synergy of the combination therapy of ganetespib with docetaxel in the treatment of prostate cancer (FIGS. 13-17).

All publications, patent applications, patents, and other documents cited herein are incorporated by reference in their entirety. In case of conflict, the present specification, including definitions, will control. In addition, the materials, methods, and examples throughout the specification are illustrative only and not intended to be limiting in any way.

What is claimed is:

1-22. (canceled)

23. A method for treating a subject with prostate cancer, comprising administering to the subject an amount of from about 2 mg/m² to about 260 mg/m² of the triazolone compound of 3-(2,4-dihydroxy-5-isopropyl-phenyl)-4-(1-methyl-indol-5-yl)-5-hydroxy-[1,2,4]triazole, or 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof.

24. The method of claim 23, wherein the subject with prostate cancer was previously treated with docetaxel-based chemotherapy.

25. The method of claim 23, wherein the amount of the triazolone compound administered is from about 75 mg/m² to about 260 mg/m².

26. The method of claim 25, wherein the amount of the triazolone compound administered is from about 100 mg/m² to about 260 mg/m².

27. The method of claim 26, wherein the amount of the triazolone compound administered is from about 125 mg/m² to about 260 mg/m².

28. The method of claim 27, wherein the amount of the triazolone compound administered is from about 150 mg/m² to about 260 mg/m².

29. The method of claim 28, wherein the amount of the triazolone compound administered is from about 175 mg/m² to about 260 mg/m².

30. The method of claim 23, wherein the amount of the triazolone compound administered is about 75 mg/m², about 85 mg/m², about 100 mg/m2, about 110 mg/m², about 115 mg/m², about 120 mg/m², about 145 mg/m², about 150 mg/m², about 175 mg/m², about 180 mg/m², about 215 mg/m² or about 260 g/m².

31. The method of claim 23, wherein the triazolone compound is administered by intravenous infusion.

32. The method of claim 31, wherein the infusion is a peripheral intravenous infusion.

33. The method of claim 32, wherein the triazolone compound is infused over 60 minutes.

34. The method of claim 23, further comprising administering one or more additional therapeutic agents.

35. The method of claim 34, wherein the additional therapeutic agent is a paclitaxel analogue.

36. The method of claim 35, wherein the paclitaxel analogue is docetaxel.

37. A method of treating a subject with prostate cancer, comprising administering to the subject an effective amount of docetaxel and an effective amount of the triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof.

38. A method of treating a subject with prostate cancer, comprising administering to the subject an effective amount of docetaxel and a synergistic amount of the triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof.

39. A method of treating a subject with prostate cancer, comprising administering to the subject a synergistic amount of docetaxel and a synergistic amount of the triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof.

40. The method of claim 23, wherein the prostate cancer is metastatic prostate cancer, metastatic hormone-resistant prostate cancer, or metastatic castration-resistant prostate cancer.

41. The method of claim 40, wherein the prostate cancer was previously treated with docetaxel-based chemotherapy.
